(19) United States
(12) Patent Application Publication
(10) Pub. No.: US 2011/0118464 A1
(43) Pub. Date: May 19, 2011

(54) HALOGENATED XANTHINE DERIVATIVES AND PRECURSORS THEREOF FOR ANTI-CANCER AND ANTI-METASTASIS ACTIVITY AND PREPARING METHOD THEREOF

(75) Inventor: Ing-Jun Chen, Linya District (TW)

(73) Assignee: Kaohsiung Medical University

(21) Appl. No.: 12/621,006

(22) Filed: Nov. 18, 2009

(51) Int. Cl. C07D 473/08; C07D 241/04

(52) U.S. Cl. 544/272; 544/267; 544/395

(57) ABSTRACT

The halogenated xanthine derivatives and the precursors thereof for anti-cancer and anti-metastasis activity and the preparation method thereof are provided. The halogenated xanthine derivatives can further be radio-labeled with the radioactive halide group. The growth of human prostate carcinoma, tongue squamous cell carcinoma, colon adenocarcinoma and lung carcinoma can be inhibited and arrested in G₀/G₁ phase by KMUP-2Cl (7-[2-[4-(chlorophenyl)-piperazinyl]ethyl]-1,3-dimethyl-8-chloroxanthine). In addition, the growth of human prostate carcinoma LNCaP can be inhibited by KMUP-1 (7-[2-[4-(2-chlorobenzene)-piperazinyl]-ethyl]-1,3-dimethyl-xanthine), and the LNCaP prostate cancer xenograft growth in nude mice is effectively inhibited by the intraperitoneal injection and per oral administration of KMUP-1.

### FIELD OF THE INVENTION

The present invention relates to the halogenated xanthine derivatives and the preparing method thereof. In particular, the present invention relates to the halogenated xanthine derivatives for anti-cancer and anti-metastasis and the preparing method thereof, and the halogenated xanthine derivatives further can be labeled with isotopic halogens.

### BACKGROUND OF THE INVENTION

The growth and proliferation of the prostate carcinoma relate to the gene regulations of androgen and androgen receptor (AR). Cai et al. (2007) reported that the α1-subunit of solubale guanylyl cyclase (sCG), composed of an α-subunit and a β-subunit, is a new androgen regulation gene. Solubale guanylyl cyclase wildly regulates the cellular function of nitric oxide and plays an important role in signaling transduction in animals and plants (Krumenacker et al., 2004). Nitric oxide combines with and activates sGC resulting in guanosine 5′-triphosphate (GTP) converted as 3′,5′-cyclic guanosine monophosphate (cGMP). Next, cGMP activates a serious of proteins, including ion channels, protein kinases and phosphodiesterase (PDE) (Papandreou et al., 1998). Androgen can stimulate sGCα1 expression in the prostate carcinoma. If sGCα1 expression is ceased, androgen-dependent and androgen-independent AR-positive receptors will stimulate the growth of the prostate carcinoma, and the individual sGCα1 over-expression will stimulate the proliferation of prostate carcinoma.

The interaction of cyclic adenosine monophosphate (cAMP)/protein kinase A (PKA) and Rho-A-participated cell signaling play the important role in the human prostate carcinoma PC-3 (Chen et al., 2005). In the human prostate carcinoma PC-3, cAMP/PKA can stimulate the Rho-A activation, and serine¹⁸⁸ phosphorylation of Rho-A is necessary to the Rho-A activation. Since Rho-A participates the rearrangement of actin cytoskeleton, including cell attachment and movement, the interaction of cAMP/PKA and Rho-A cell signaling will change the cellular morphologyand the changes of cytoskeleton, migration and anchorage-independent growth.

In addition, the regulation pathways of sGC/cGMP/protein kinase G (PKG) and Rho kinase (ROCK2) are important on prostate smooth muscle tension. The inventor of the present invention found that PDE5A/ROCK2 inhibitor, 7-[2-[4-(2-chlorobenzene)piperazinyl]ethyl]-1,3-dimethylxanthine (abbreviated as KMUP-1), can inhibit the growth of the human prostate epithelial carcinoma PZ-HPV-7 (Liu et al., 2007). KMUP-1 is a synthetic xanthine derivative. In the past researches, it has been proved that KMUP-1 can increase the amount of cyclic nucleotide, inhibit PDE, activate potassium ion channel resulting in relaxations in aortic (Wu et al., 2001), corporeal carvenosa (Lin et al., 2002) and tracheal smooth muscles (Wu et al., 2004). KMUP-1 inhibits the phenylephrine-induced contraction, has effective inhibition activity of α_{1A}/α_{1D}-adenoreceptor combination, increases the amount of cAMP/cGMP, and increases phenylephrine-induced ROCK2 expression. KMUP-1 can inhibit the cellular growth of PZ-HPV-7 cells resulting in the cease of cell cycle at G0/G1 phase and increase p21 expression. The inventor of the present invention proved the knowledge of sGC/cGMP/PKG and ROCK2 regulation on the relaxation and proliferation of prostate. However, the anti-tumor and anti-tumor metastatic mechanisms of the prostate epithelial carcinoma are unknown (Liu et al., 2007).

It is therefore attempted by the applicant to deal with the above situation encountered in the prior art.

### SUMMARY OF THE INVENTION

In accordance with one aspect of the present invention, a halogenated xanthine derivative is provided in the present invention. The halogenated xanthine derivative has a structure as shown in the following formula I.

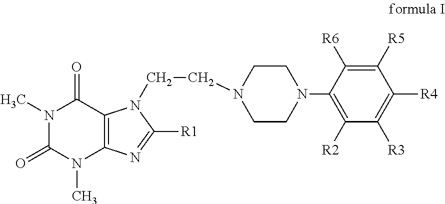

R1 represents a first substituted group selected from a group consisting of a first hydrogen group, a first halide group and a C₁-C₁₀ alkyl group, and each of R2, R3, R4, R5 and R6 represents a second substituted group being one of a second hydrogen group and a second halide group. Each of the first halide group and the second halide group has one halide atom selected from a group consisting of a chloride (Cl), a bromide (Br) and an iodine (I).

Preferably, R1, R2, R3, R4, R5 and R6 have an identical radioactive halide group.

Preferably, the identical radioactive halide group is one radioactive halide atom selected from a group consisting of a radioactive chloride, a radioactive bromide and a radioactive iodine.

Preferably, R1, R2, R3, R4, R5 and R6 are different radioactive halide groups.

Preferably, each of the different radioactive halide groups is one radioactive halide atom selected from a group consisting of a radioactive chloride, a radioactive bromide and a radioactive iodine.

Preferably, each of R1, R2, R3, R4, R5 and R6 is a radioactive halide group selected from a group consisting of a ³⁸Cl, a ³⁷Cl, a ⁷⁵Br, a ⁷⁶Br, a ⁷⁷Br, a ⁸²Br, a ¹²²I, an ¹²³I, an ¹²⁴I, an ¹²⁵I and an ¹³¹I.

Preferably, the halogenated xanthine derivative has a function selected from a group of inhibiting a cancer, inhibiting a cancer metastasis and a combination thereof.

In accordance with another aspect of the present invention, a method for preparing a halogenated xanthine derivative is provided. The halogenated xanthine derivative has a structure as shown in the following formula II.

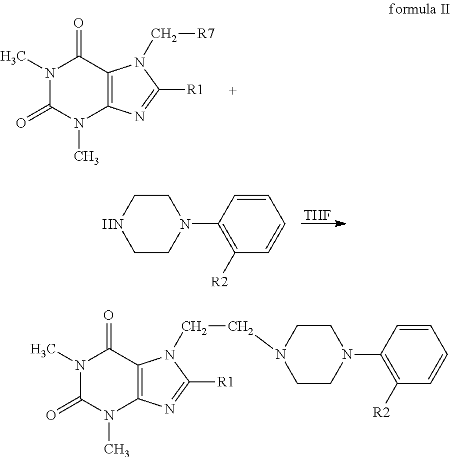

the method include a step of (a) reacting a theophylline with a piperazine in a first solution to obtain the halogenated xanthine derivative. R1 represents a first substituted group being one of a first hydrogen group and a first halide group, R7 represents a second substituted group being one of a second hydrogen group and a second halide group, and R2 represents a third substituted group being one of a third hydrogen group and a third halide group.

Preferably, the method further includes a step of: (b) radio-labeling a radioactive halogen group from a radioactive halide compound on at least one of R1, R2 and R7 of the halogenated xanthine derivative to obtain a radioactive halogenated xanthine derivative.

Preferably, the radioactive halide compound is one of a radioactive halogen, a radioactive sodium halide and a combination thereof which are dissolved in a second solution.

Preferably, the second solution is an organic solvent selected from a group consisting of a tetrahydrofuran, a methanol and an ethanol.

Preferably, the first solution is an organic solvent selected from a group consisting of a tetrahydrofuran, a methanol and an ethanol.

In accordance with another aspect of the present invention, a precursor of a halogenated xanthine derivative is provided. The precursor has a structure as shown in the following formula III.

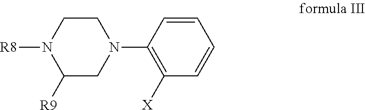

R8 represents a first substituted group selected from a group consisting of a first hydrogen group, a first C₁-C₁₂ alkyl group, a first C₁-C₁₂ alkenyl group and a first C₁-C₁₂ dihaloalkyl group, R9 represents a second substituted group selected from a group consisting of: a second hydrogen group, a second C₁-C₁₂ alkyl group, a second C₁-C₁₂ alkenyl group, a monohaloalkyl group and a second C₁-C₁₂ dihaloalkyl group, and X represents one of a halide group and a radioactive halide group.

Preferably, the halide group is a halide atom selected from a group consisting of a chloride (Cl), a bromide (Br) and an iodine (I), and the radioactive halide group is a radioactive halide atom selected from a group consisting of a radioactive chloride, a radioactive bromide and a radioactive iodine.

In accordance with another aspect of the present invention, a precursor of a halogenated xanthine derivative is provided. The precursor has a structure as shown in the following formula IV.

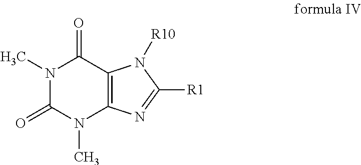

R1 represents a first substituted group being one of a hydrogen group and a halide group, and R10 represents a C₁-C₁₂ iodoalkyl group.

Preferably, the halide group is one halide atom selected from a group consisting of a chloride (Cl), a bromide (Br) and an iodine (I).

Preferably, the precursor is obtained by reacting a dihaloalkane with one of a theophylline and a chlorotheophylline.

Preferably, an iodide group of the C₁-C₁₂ iodoalkylgroup of the precursor is further radio-labeled a first radioactive halide group.

Preferably, the halide group is further radio-labeled a second radioactive halide group when R1 of the precursor is the halide group.

Preferably, the precursor is an iodomethyl chlorotheophylline when R1 is the chloride group and R10 is an iodomethyl group.

The halogenated xanthine derivative and the preparing method thereof in the present invention, especially relative to the halogenated xanthine derivative for anti-cancer and anti-metastasis and the preparing method thereof, further can be labeled the radioactive halogen thereon. The prepared radioactive pharmaceuticals not only use in clinical diagnosis and clinically radioactive reagent, but also use in labeled pharmaceuticals.

The effect of detection and quantity can be easily achieved using the radioactivity of radioactive isotope and radioactive isotope detector. For instance, radioactive iodine (¹³¹I, ¹²⁵I) has been widely used in the detection of radioactive material in the clinical diagnosis and treatment. With the design and determination of computer software, medical images progress from the present two-dimensional image to three-dimensional image and the information observation such as image variation. The halogenated xanthine derivative in the present invention selects different halogenated radioactive element, such as ³⁸Cl, ³⁷Cl, ⁷⁵Br, ⁷⁶Br, ⁷⁷Br, 82Br, ¹²²I, ¹²³I, ¹²⁴I, ¹²⁵I, and ¹³¹I, simultaneously on the substituted group of different position, so as to improve detection, diagnostic tracing and treatment of anti-cancer.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 illustrates the relationship between the survival rate and the various concentrations of KMUP-2Cl while the human normal prostate cell line PZ-HPV-7 is treated with KMUP-2Cl for 24 and 48 hours respectively;

FIGS. 2(A) and 2(B) illustrate the relationships between the survival rate and the various concentrations of (A) KMUP-2Cl and (B) KMUP-1 respectively while the human tongue squamous cell carcinoma SCC-25 is treated with (A) KMUP-2Cl or (B) KMUP-1 for 24 and 48 hours respectively;

FIGS. 3(A) and 3(B) illustrate the relationship between the survival rate and the various carcinoma cell lines while these carcinoma cell lines respectively are treated with 100 μM KMUP-2Cl for 24 and 48 hours;

FIGS. 4(A) and 4(B) illustrate the relationships between the apoptotic induction effect and the various concentrations of KMUP-2Cl while the human tongue squamous carcinoma SCC-25 is treated with KMUP-2Cl for (A) 24 hours and (B) 48 hours respectively;

FIGS. 5(A) and 5(B) illustrate the relationship between the growth inhibition and the various concentrations of KMUP-2Cl when the human tongue squamous carcinoma SCC-25 is treated with KMUP-2Cl for (A) 24 hours and (B) 48 hours respectively;

FIGS. 6(A) and 6(D) illustrate the distributions of different phases of SCC-25 when the human tongue squamous carcinoma SCC-25 is treated with various concentrations of KMUP-2Cl for (A, B) 24 hours and (C, D) 48 hours respectively;

FIGS. 7(A) to 7(C) illustrate the relationships between the survival rate and various concentrations of KMUP-1 while the human prostate carcinomas (A) LNCaP, (B) DU145 and (C) PC-3 respectively treated with various concentrations of KMUP-1 for 24 and 48 hours;

FIGS. 8(A) and 8(B) illustrate the distributions of the cell cycle in LNCaP while the human prostate carcinoma LNCaP is treated with various concentrations of KMUP-1 for (A) 24 hours and (B) 48 hours respectively;

FIG. 9(A) illustrates the relationships between the drug concentrations and the cGMP (3′,5′-cyclic guanosine monophosphate) concentration, in which the human prostate epithelial carcinoma LNCaP is treated with various concentrations of KMUP-1 individually for 48 hours, and treated with 1 μM solubale guanylyl cyclase (sGC) inhibitor ODQ and various concentrations of KMUP-1 together for 48 hours;

FIG. 9(B) illustrates the relationships between the drug concentrations and the cAMP (cyclic adenosine monophophate) concentration, in which the human prostate epithelial carcinoma LNCaP is treated with 10 μM sGC inhibitor SQ22536, various concentrations of KMUP-1, and 100 μM KMUP-1 plus 10 μM sGC inhibitor SQ22536 respectively for 48 hours;

FIGS. 10(A) and 10(B) illustrate (A) the immunoblotting analysis of AR (androgen receptor) and (B) the relationship between the drug concentrations and dihydrotestosterone receptor, in which the human prostate epithelial carcinoma LNCaP is treated with various concentrations of KMUP-1 and KMUP-1 vehicle for 48 hours;

FIGS. 11(A) to 11(D) illustrate (A, C) the immunoblotting analysis of p21 protein (A: 24 hours; C: 48 hours) and (B, D) the relationship between the relative intensity of p21 expression and various concentrations of KMUP-1 (B: 24 hours; D: 48 hours), in which the human prostate epithelial carcinoma LNCaP is treated with various concentrations of KMUP-1 for 24 and 48 hours respectively;

FIG. 12(A) to 12(D) illustrate (A, C) the immunoblotting analysis of p27 protein (A: 24 hours; C: 48 hours) and (B, D) the relationship between the relative intensity of p27 expression and various concentrations of KMUP-1 (B: 24 hours; D: 48 hours), in which the human prostate epithelial carcinoma LNCaP is treated with various concentrations of KMUP-1 for 24 and 48 hours respectively;

FIGS. 13(A) to 11(D) illustrate (A, C) the immunoblotting analysis of Cyclin D protein (A: 24 hours; C: 48 hours) and (B, D) the relationship between the relative intensity of Cyclin D expression and various concentrations of KMUP-1 (B: 24 hours; D: 48 hours), in which the human prostate epithelial carcinoma LNCaP is treated with various concentrations of KMUP-1 for 24 and 48 hours respectively;

FIG. 14(A) to 14(D) illustrate (A, C) the immunoblotting analysis of CDK4 protein (A: 24 hours; C: 48 hours) and (B, D) the relationship between the relative intensity of CDK4 expression and various concentrations of KMUP-1 (B: 24 hours; D: 48 hours), in which the human prostate epithelial carcinoma LNCaP is treated with various concentrations of KMUP-1 for 24 and 48 hours respectively;

FIG. 15(A) to 15(D) illustrate (A, C) the immunoblotting analysis of CDK6 protein (A: 24 hours; C: 48 hours) and (B, D) the relationship between the relative intensity of CDK6 expression and various concentrations of KMUP-1 (B: 24 hours; D: 48 hours), in which the human prostate epithelial carcinoma LNCaP is treated with various concentrations of KMUP-1 for 24 and 48 hours respectively;

FIGS. 16(A) to 16(C) illustrate the relationships between the various concentrations of KMUP-1 and (A) Bax expression, (B) Bcl-2 expression, and (C) the Bax/Bcl-2 ratio, in which the human prostate epithelial carcinoma LNCaP is treated with various concentrations of KMUP-1 for 48 hours;

FIG. 17(A) illustrates the relationship between the tumor size and the experimental time (week), in which LNCaP-xenografted rude mice are injected subcutaneously with KMUP-1;

FIG. 17(B) illustrate the relationship between the body weight and the experimental time (week), in which LNCaP-xenografted rude mice are injected subcutaneously with KMUP-1;

FIG. 18(A) illustrates the relationship between the tumor size and the experimental time (week), in which LNCaP-xenografted rude mice are orally dosed with KMUP-1; and

FIG. 18(B) illustrate the relationship between the body weight and the experimental time (week), in which LNCaP-xenografted rude mice are dosed orally with KMUP-1.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### Embodiment 1: Synthesis of KMUP-1

Twenty grams of 7-ethylchloro-theophylline is added into a flask containing 50 ml of tetrahydrofuran (THF) and 60 ml of 1-(2-chlorophenyl)-piperazine, and a suitable amount of sodium carbonate (Na₂CO₃) is added therein. The flask is connected with a condensing tube, and the reaction mixture in the flask is heated to boil to reflux for 2 hours. The reaction mixture is cooled after the reaction is completed, the product is dissolved and eluted with ethanol, and the elutant is collected, concentrated and heated under the water bath. The elutant is filtered while heated, and the filtered solution is cooled and the white crystal product is obtained. The white crystal product is 7-[2-[4-(2-chlorobenzene)piperazinyl]ethyl]-1,3-dimethylxanthine (abbreviated as KMUP-1). The reaction formula is illustrated as follows. In addition, tetrahydrofuran can be substituted for methanol or ethanol.

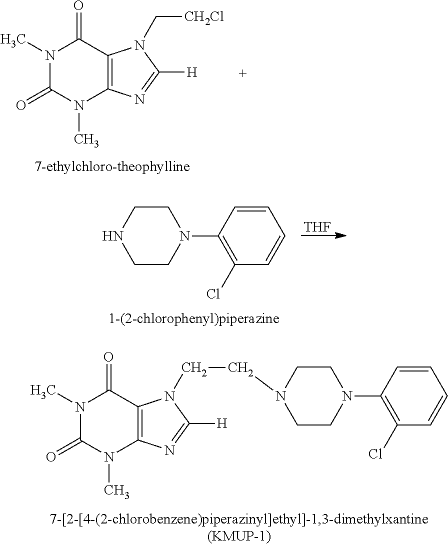

### Embodiment 2: Synthesis of KMUP-H

Twenty grams of 2-ethylbromo-theophylline is added into a flask containing 50 ml of tetrahydrofuran and 60 ml of 1-phenylpiperazine, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, and the filtered solution is cooled and the white crystal product is obtained. The white crystal product is 7-[2-[4-(2-phenyl)-piperazinyl]ethyl]-1,3-dimethylxanthine (abbreviated as KMUP-H), and the chemical formula is illustrated as follows.

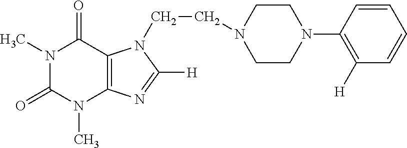

### Embodiment 3: Synthesis of KMUP-2Cl

Twenty grams of 7-ethylbromo-8-chloro-theophylline is added into a flask containing 50 ml of tetrahydrofuran and 60 ml of 1-(2-chlorophenyl)piperazine, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, and the filtered solution is cooled and the white crystal product is obtained. The white crystal product is 7-[2-[4-(2-chlorophenyl)-piperazinyl]ethyl]-1,3-dimethyl-8-chloroxanthine (abbreviated as KMUP-2Cl), which has a chemical formula as follows.

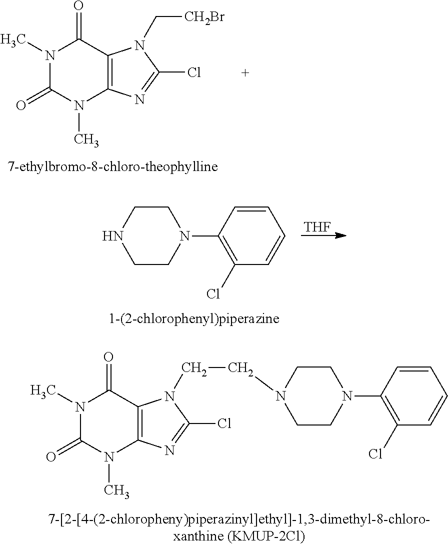

### Embodiment 4: Synthesis and Radio-Labeling of KMUP-I

1. Twenty grams of 2-ethylbromo-theophylline is added into a flask containing 50 ml of tetrahydrofuran and 60 ml of 1-(2-iodophenyl)piperazine, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, and the filtered solution is cooled and the white crystal product is obtained. The white crystal product is KMUP-I, which has a structural formula as follows.

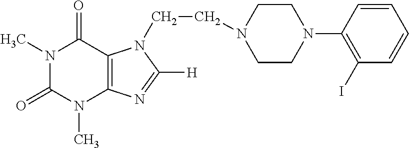

2. Twenty grams of KMUP-H is added into a flask containing 50 ml of tetrahydrofuran and 60 mg of iodine (I₂), and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, and the filtered solution is cooled and the white crystal product is obtained. Iodine can be substituted as the mixture of potassium iodide (KI) and I₂ dissolved in THF. The reaction formula of KMUP-I is illustrated as follows.

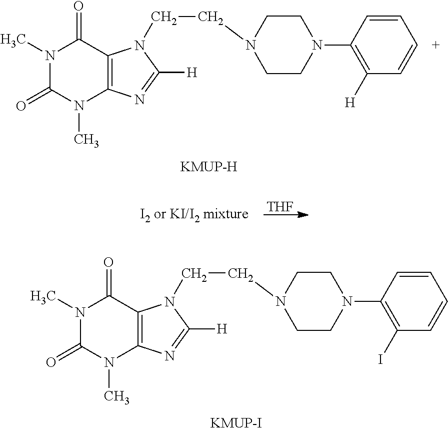

3. The equal amount of non-radioactive KMUP-I and radioactive sodium iodide (NaI*) are mixed in tetrahydrofuran to exchange the radioactivity with each other, so as to obtain the isotope-labeled KMUP-I (KMUP-I*).

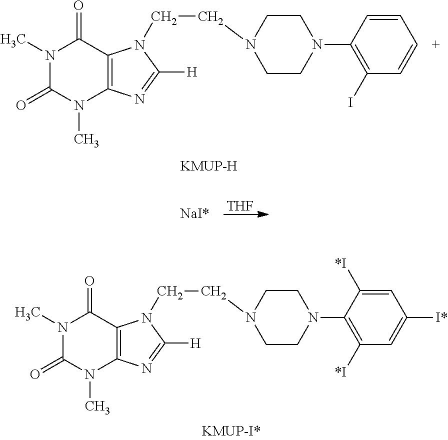

### Embodiment 5: Synthesis of KMUP-Br

1. Twenty grams of 2-propylbromo-theophylline is added into a flask containing 50 ml of tetrahydrofuran and 60 ml of 1-(3-chlorophenyl)piperazine, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, the filtered solution is cooled, and the white crystal product is obtained.

2. Twenty grams of theophylline is added into a flask containing 50 ml of tetrahydrofuran and 60 ml of 1-(3-chlorophenyl)-4-(3-chloroproyl)-piperazine HCl, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, the filtered solution is cooled, and the white crystal product is obtained.

### Embodiment 6: Synthesis of KMUP-12Cl

Twenty grams of KMUP-2Cl is added into a flask containing 50 ml of tetrahydrofuran and 60 ml of I₂, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, the filtered solution is cooled, and the yellow crystal product is obtained. Iodine (I₂) can be substituted for the mixture of potassium iodide (KI) and I₂ dissolved in tetrahydrofuran.

### Embodiment 7: Synthesis and Radio-Labeling of Radioactive KMUP-12Cl

1. Twenty grams of KMUP-2Cl is added into a flask containing 50 ml of tetrahydrofuran and 60 ml of radioactive I₂, and a suitable amount of sodium carbonate is added therein. The flask is connected with a condensing tube, and the reaction mixture in the flask is heated to boil to reflux for 2 hours. The reaction mixture is cooled after the reaction is completed, and the product is dissolved and eluted with ethanol. The elutant is collected, concentrated and heated under the water bath. The elutant is filtered while heating, the filtered solution is cooled, and the yellow crystal product is obtained. Iodide (I₂) can be substituted for the mixture of potassium iodide (KI) and I₂ dissolved in tetrahydrofuran.

2. The equal amount of non-radioactive KMUP-I2Cl and radioactive sodium iodide (NaI) are mixed in tetrahydrofuran to exchange the radioactivity with each other, so as to obtain the radio-labeled KMUP-I2Cl (KMUP-I2Cl*).

### Embodiment 8: Structure of Precursor of Halogenated Xanthine Derivative

The precursor of the halogenated xanthine derivative for anti-cancer is provided in the present invention, and the precursor thereof has a structural formula as follows, in which R8 is a substitutive group selected from a group consisting of a hydrogen group (H), an alkyl group (CₙH₂ₙ₊₁), an alkenyl group and a dihaloalkyl group. The number of carbon atom of the alkyl group, the alkyenyl group and the dihaloalkyl group is ranged between 1 and 12 (1≦n≦12), R9 is one of a hydrogen group, an alkyl group, an alkenyl group, a monohaloalkyl group and a dihaloalkyl group. The number of carbon atom of the hydrogen group, the alkyl group, the alkenyl group, the monohaloalkyl group and the dihaloalkyl group is ranged between 1 and 12 (1≦n≦12). X is one substitutive group selected from a group consisting of a halide group (X) and a radioactive halide group (X*). The halide group includes one of a chloride and an iodide, and the radioactive halide group includes a radioactive chloride and an radioactive iodide.

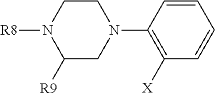

The precursors of the halogenated xanthine derivatives in the present invention includes at least two chemicals nominated as [1-alkyl,4-(2-halophenyl)]piperazine and [1(2-halophenyl),4-methyl]piperazine, such as 2-iodophenyl piperazine and [1(2-iodophenyl),4-methyl]piperazine.

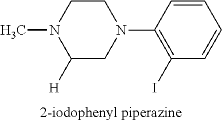

### Embodiment 9: Structure of Precursor of Halogenated Xanthine Derivatives

The precursor of the halogenated xanthine derivative for anti-cancer is further provided in the present invention, and the precursor has a structural formula as follows, in which R1 is one of a hydrogen group (H) and a chloride group (Cl), and R10 is one substitutive group selected from a group consisting of an iodoalkyl group (CₙH₂ₙI) which the number of carbon atom is ranged between 1 and 12 (1≦n≦12). The precursor is obtained by reacting dihaloalkane with theophylline or chlorotheophylline.

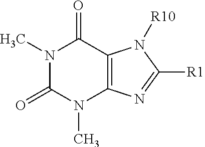

The chemical formula of one of the precursor in this embodiment is iodomethyl chlorotheophylline (iodocaffeine), which has a structural structure as follows.

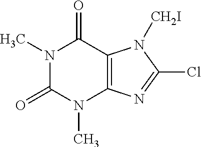

The precursor in this embodiment can be radio-labeled using radioactive halogen, such as the radio-labeled iodomethyl chlorotheophylline, which is shown as the following structural formula.

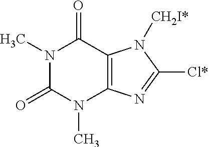

### Biological Experiment 1: MTT Assay

Cells at a density of 10⁵ cells/well are seeded in 24-well cell culture plate to 90% confluence, and KMUP-2Cl or KMUP-1 at different concentrations (0.1, 1, 10 and 100 μM) is added therein. The cells are incubated at 37° C. and an atmosphere of 5% CO₂ for 24 and 48 hours, then the medium is replaced with 490 μl of fresh medium and 10 μl of 5 mg/ml of methylthiazolyldiphenyl-tetrazolium bromide (MTT). After the reaction mixture is mixed well, the cell culture plate is covered with the aluminum foil and further is incubated in the incubator for 2.5 hours. The MTT solution is withdrawn while the reaction time is finished, 500 μl of acidified isopropanol is added into the well to dissolve the crystal violet, formazan. Two hundred microliter of the supernatant is transferred to another new 96-well cell culture 10 minutes later to determine the absorbance at the wavelengths of 540 nm (OD₅₄₀) and 630 nm (OD₆₃₀) using spectrophotometer (Hitachi U-200, Japan). The drug-treated cell survival rate is evaluated by comparing the value of OD₅₄₀-OD₆₃₀ in each group with that in the control group. The cell survival rate represents the toxicity of the drug to the cells.

### Biological Experiment 2: Trypan Blue Exclusion Assay

Tongue squamous cell carcinoma SCC-25 (10⁵ cells/well) is seeded in 6-well cell culture plate to 90% confluence, then KMUP-2Cl at different concentrations (0.1, 1, 10 and 100 μM) respectively is added therein. The cells are incubated at 37° C. and an atmosphere of 5% CO₂ for 24 and 48 hours. The cell survival rate is determined by trypan blue exclusion assay. Firstly, 20 μl cell supernatant is mixed with 20 μl of trypan blue, then 15 μl mixture is added into the groove of the hemocytometer which has a cover lid thereon. The cells are counted under the microscope, and the live cells are not stained but the dead ones are blue. The total cells in four squares are counted, then the numbers is divided by four, multiplied with the dilution rate (at least 2 because of mixing with the equal volume of trypan blue). Finally, the number is multiplied with 10⁴, and the result is the total cell numbers in the cell supernatant per milliliter.

### Biological Experiment 3: Analysis of Cell Cycle

The counted cells (10⁵ cells/ml×3 ml) are seeded in the 6-well cell culture plate for 24 hours for attaching on the bottom of the cell culture plate, then DMEM/F12 medium without fetal bovine serum (FBS) is added therein for further incubation for 24 hours. Next, the medium is replaced with DMEM/F12 supplemented with 10% FBS, and the drugs at different concentrations are respectively added therein for reacting 24 and 48 hours. The cellular inoculate is harvested, and the wells are washed with phosphate buffered saline (PBS) for twice or triplet. The cells are eluted with 0.25% trypsin-0.02% EDTA, and are collected with the original inoculate in the centrifuge tube which is centrifuged at 4° C. at 1250×g (1200 rpm) for 5 minutes. Next, the supernatant is discarded and the pellet is resuspended in 1 ml PBS and the resuspended cells are transferred to an eppendorf tube which is centrifuged once again. The supernatant is discarded, and the cells are fixed with 300 μl PBS (4° C.) and 700 μl of 99.5% ethanol (50, 50, 100, 100, 200 and 200 μl ethanol respectively and sequentially added to make the cells resuspended in the well) for 30 minutes. The supernatant is discarded after centrifuging, then 570 μl PBS, 2 μl of 10 μg/ml ribonuclease (RNase) and 30 μl of 0.5% Triton are added therein and reacted at 37° C. for 1 hour. The supernatant is discarded after centrifuging, then 600 μl PBS and 1 μl of 10 μg/ml propidium iodide (PI) are added therein. The tube is rotated at 4° C. for 15 to 30 minutes on the DS LAB Rotator. The cells are transferred into a tube, and the cells are detected using CyFlow® cytometer. The ratios of G₀/G₁, S and G₂/M phases are determined using multicycle DNA analysis software.

### Biological Experiment 4: Protein Assay

The amount of protein is determined by Bio-Rad protein reagent. Bovine serum albumin (BSA) of 0.1 mg/ml is the protein standard in the protein assay. Distilled water of 240 μl containing various known protein standards (the amounts of protein respectively are 0, 2, 4, 8, 12, 16, 20 and 30 μg) and 60 μl of Bio-Rad reagent are mixed gently in 96-well cell culture plate. The absorbance at the wavelength of 595 nm relative to the absorbance of blank test is determined using spectrophotometer (Hitachi U-200, Japan). A standard curve is evaluated from the known amounts of the protein standards and the absorbance thereof, and the amounts of the unknown proteins can be obtained according to this standard curve.

### Biological Experiment 5: Protein Expression

The quantified proteins are resolved by sodium dodecyl sulfate-polyacrylamide gel electrophoresis (SDS-PAGE), and the protein expression is evaluated by immunoblotting. The dilution rate of primary antibodies (i.e. anti-AR, anti-p21, anti-p27, anti-cyclin D, anti-cdk4 and anti-cdk6) is 1000 times, and that of anti-β-actin is 5000 times. The primary antibody detects the proteins for 1 hour, and the primary antibody is detected with the horseradish peroxidase (HRP)-conjugated secondary antibody for 1 hour. Finally, the detected protein are developed with enhanced chemical luminescence.

### Biological Experiment 6: Statistic Analysis

1. All the abovementioned experiments are at least repeated independently for triplet, and the results are shown as mean±s.e.m. The data are analyzed by ANOVA (analysis of variance), then are analyzed with Dunnett's test. P-value is a comparison comparing with the vehicle, and p-value<0.05 means the statistic significance and is shown as a star symbol (*). The vehicle is a solution containing propylene glycol, 0.2% ethanol, 0.58% dimethyl sulfoxide (DMSO) and 0.02% hydrogen chloride.

2. The calculations of 50% effective dosage (ED₅₀) and 95% confidential interval use Litchfield and Wilcoxon method.

The cell lines utilized in the present invention are listed in Table 1.

TABLE 1
Cell lines in the present invention

| Cell type | Cell line | Accession No. |
|---|---|---|
| Human normal prostate cell line | PZ-HPV-7 | BCRC* 60136 |
| Human prostate carcinoma | LNCaP | BCRC 60088 |
| Human prostate carcinoma | DU145 | ATCC** HTB-81 |
| Human prostate carcinoma | PC-3 | BCRC 60122 |
| Human tongue squamous cell carcinoma | SCC-25 | BCRC 60516 |
| Human colon adenocarcinoma | WiDr | BCRC 60157 |
| Human lung carcinoma | A549 | ATCC CCL-185 |

*BBRC is Bioresource Collection and Research Center, Taiwan, R.O.C.
**ATCC is American Type Culture Collection.

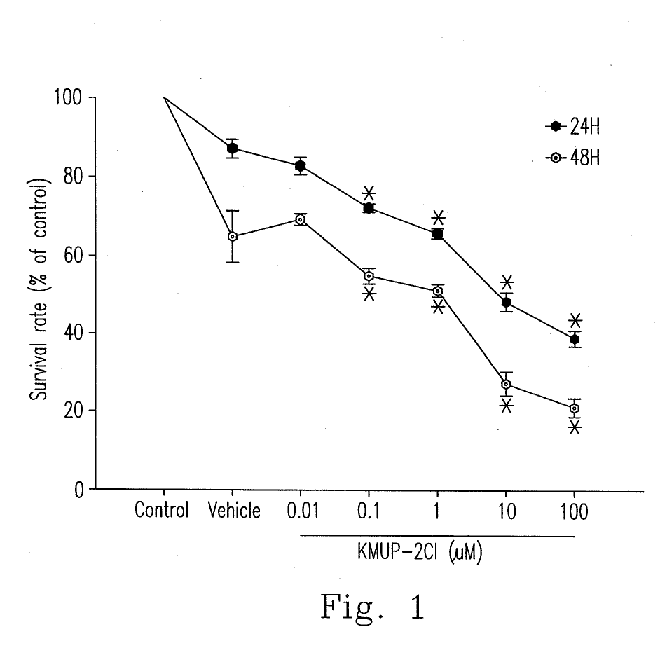

Please refer to FIG. 1, which illustrates the relationship between the survival rate and the various concentrations of KMUP-2Cl while the human normal prostate cell line PZ-HPV-7 is treated with KMUP-2Cl for 24 and 48 hours respectively. In FIG. 1, the abscissa shows the control, the vehicle, and the concentrations of KMUP-2Cl respectively, and the ordinate is the survival rate relative to the control. From the MTT assay, the survival rate of PZ-HPV-7 decreases with the increased concentrations of KMUP-2Cl, and the effect of the survival rate of KMUP-2Cl to PZ-HPV-7 increases with the increased reaction time.

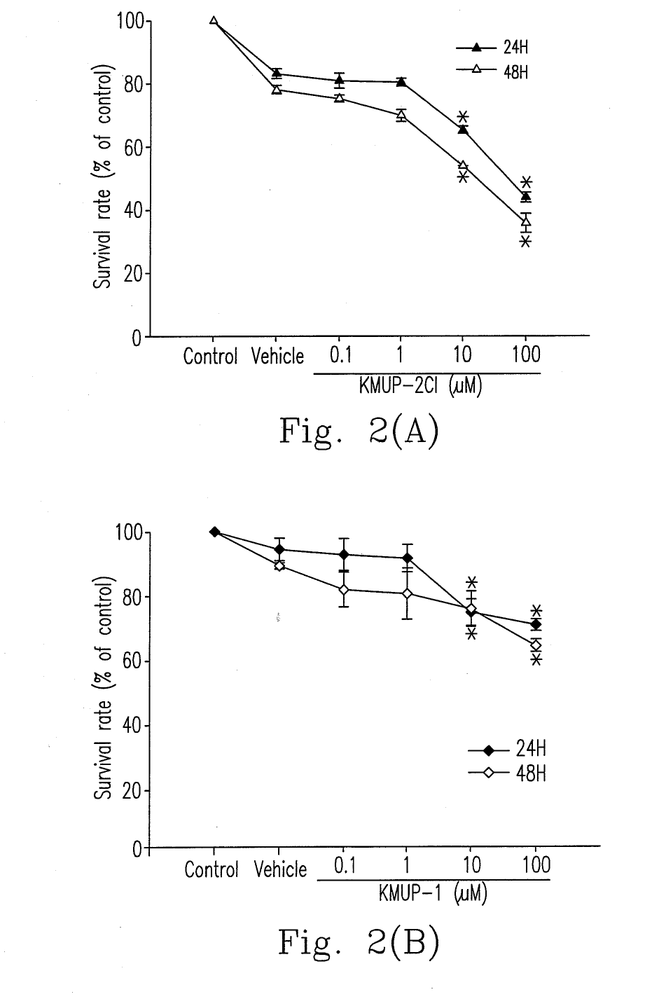

Please refer to FIGS. 2(A) and 2(B), which illustrate the relationships between the survival rate and the various concentrations of (A) KMUP-2Cl and (B) KMUP-1 respectively while the human tongue squamous cell carcinoma SCC-25 is treated with (A) KMUP-2Cl or (B) KMUP-I for 24 and 48 hours respectively. In FIG. 2(A), the abscissa shows the concentrations of KMUP-2Cl, and the ordinate is the survival rate relative to the control. The survival rate of SCC-5 decreases with the increased concentrations of KMUP-2Cl, and the effect of the survival rate of KMUP-2Cl to SCC-25 increases with the increased reaction time. In FIG. 2(B), the abscissa shows the concentrations of KMUP-1, and the ordinate is the survival rate relative to the control. The survival rate of SCC-5 decreases with the increased concentrations of KMUP-1, and the effect of the survival rate of KMUP-1 to SCC-25 increases with the increased reaction time. From the statistic results, no matter 24 or 48 hour, KMUP-2Cl inhibits more 20% SCC-25 cells than KMUP-1 at the highest concentration (100 μM) excluding any solvents effect.

From FIG. 1 and FIG. 2(A), it is known that KMUP-2Cl can inhibit the survival rate of the human normal prostate cell line PZ-HPV-7 and the human tongue squamous cell carcinoma SCC-25, and the inhibition effect of KMUP-2Cl to SCC-25 is 20% higher than that of KMUP-1 to SCC-25.

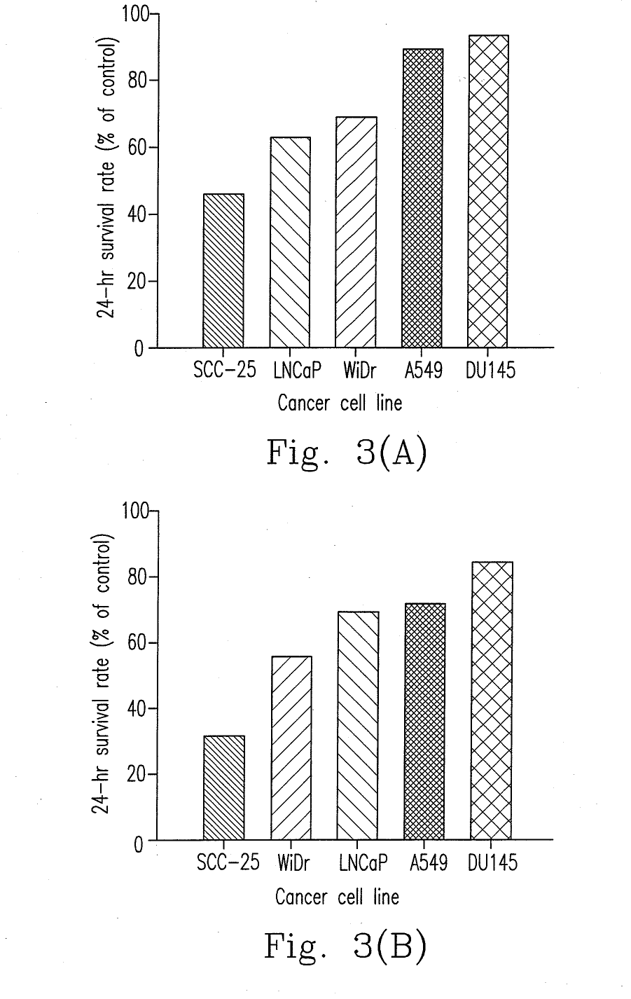

Please refer to FIGS. 3(A) and 3(B), which illustrate the relationship between the survival rate and the various carcinoma cell lines while these carcinoma cell lines respectively are treated with 100 μM KMUP-2Cl for 24 and 48 hours. The abscissas in FIGS. 3(A) and 3(B) are the various carcinoma cell lines, and the ordinates therein are the survival rates of 24 and 48 hours respectively. In FIG. 3(A), the survival rates after 100 μM KMUP-2Cl treatment for 24 hours are ranked as follows (from low to high): SCC-25, LNCaP, WiDr, A549 and DU145. However, in FIG. 2(B), the survival rates after 100 μM KMUP-2Cl treatment for 48 hours are ranked as follows (from low to high): SCC-25, WiDr, LNCap, A549 and DU145. Furthermore, comparing FIG. 3(A) with FIG. 3(B), it can be found that the survival rate of each carcinoma cell line decreases with the reaction time of KMUP-2Cl. It means that KMUP-2Cl has cytotoxicity on the human tone squamous cell carcinoma, the human prostate carcinoma, the human colon adenocarcinoma and the human lung carcinoma.

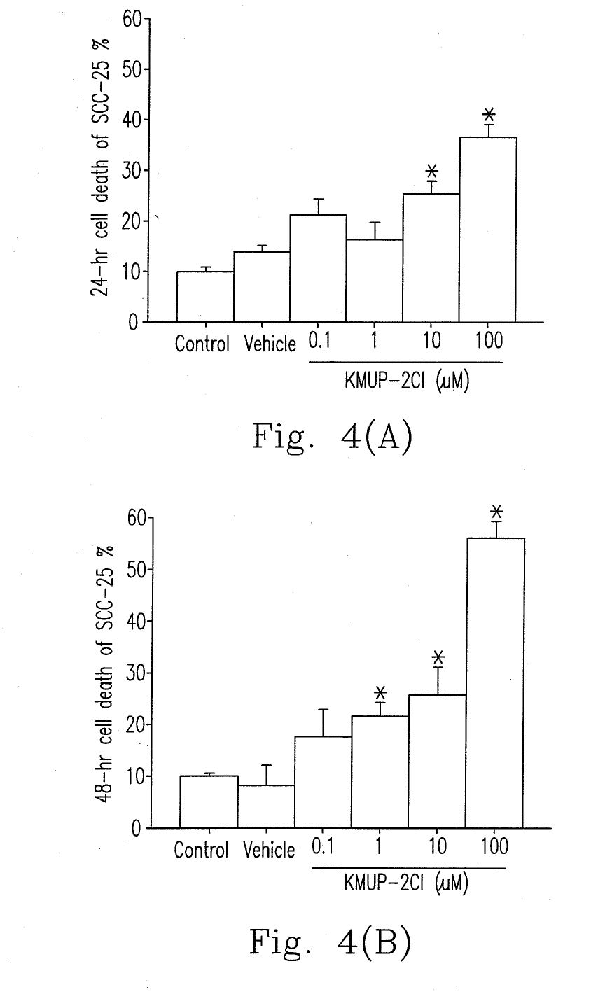

Please refer to FIGS. 4(A) and 4(B), which illustrate the relationships between the apoptotic induction effect and the various concentrations of KMUP-2Cl while the human tongue squamous carcinoma SCC-25 is treated with KMUP-2Cl for (A) 24 hours and (B) 48 hours respectively. The abscissa shows the concentrations of KMUP-2Cl, and the ordinates are (A) the 24-hour SCC-25 cell death rate and (B) the 48-hour SCC-25 cell death rate respectively. In FIG. 4(A), the cell death rate of SCC-25 increases with the increased concentrations of KMUP-2Cl after SCC-25 cells are treated with KMUP-2Cl for 24 hours. In FIG. 4(B), the cell death rate of SCC-25 also increases with the increased concentrations of KMUP-2Cl after SCC-25 cells are treated with KMUP-2Cl for 48 hours. In particular, the 48-hour cytotoxicity is significantly higher than the 24-hour cytotoxicity when KMUP-2Cl is 100 μM. After the high concentration (100 mM) of KMUP-2Cl functions for 24 and 48 hours respectively, the number of cell death significantly increases for 3.6 times (FIG. 4(A)) and 5.6 times (FIG. 4(B)).

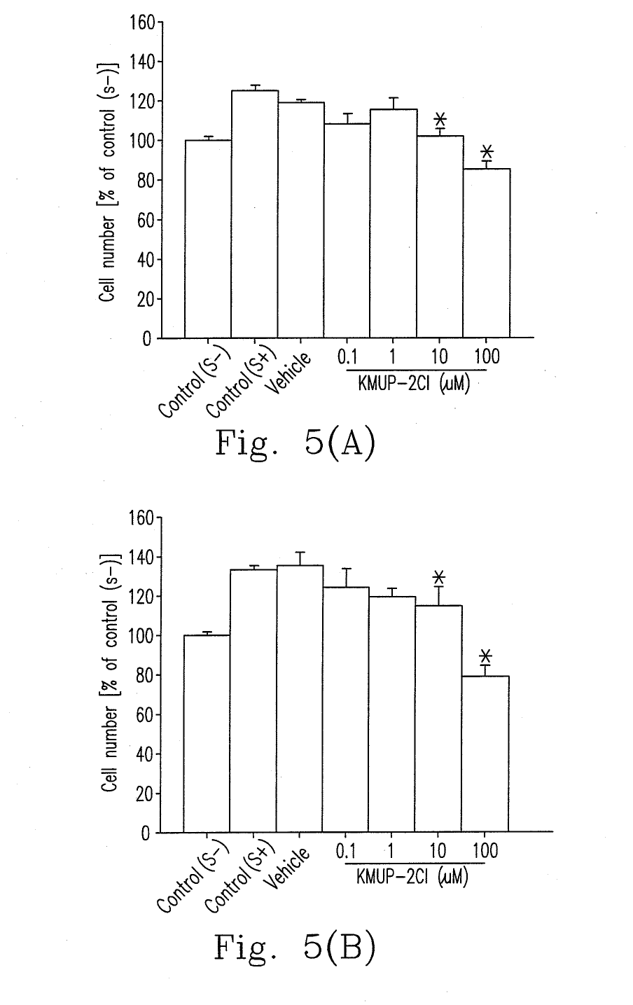

Please refer to FIGS. 5(A) and 5(B), which illustrate the relationship between the growth inhibition and the various concentrations of KMUP-2Cl when the human tongue squamous carcinoma SCC-25 is treated with KMUP-2Cl for (A) 24 hours and (B) 48 hours respectively. The abscissa shows the concentration of KMUP-2Cl, and the ordinate is the percentage of cell number relative to the control S(−). In FIGS. 5(A) and 5(B), the percentage of cell number decreases with the increased concentrations of KMUP-2Cl, and it means that the higher concentration of KMUP-2Cl induces more significant cytotoxicity to SCC-25 cells. It should be noted that the low concentrations (0.1 and 1 μM) of KMUP-2Cl would stimulate the growth of SCC-25 cells.

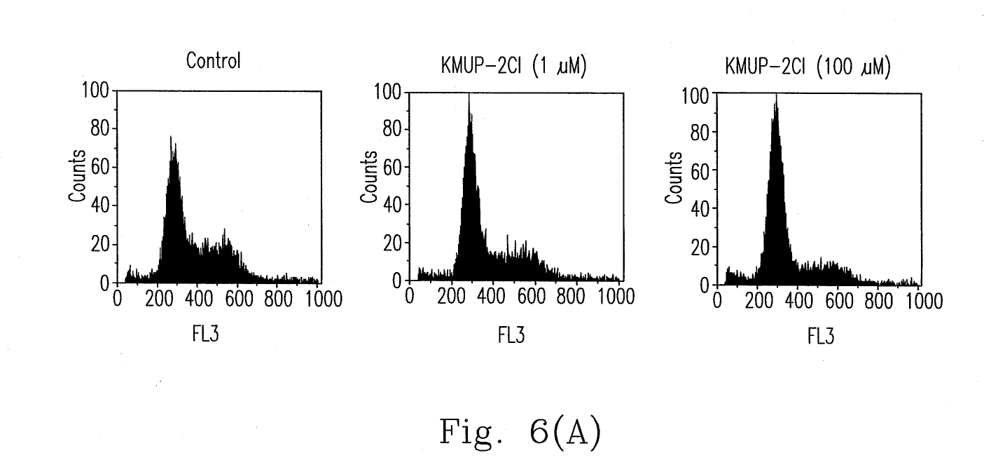

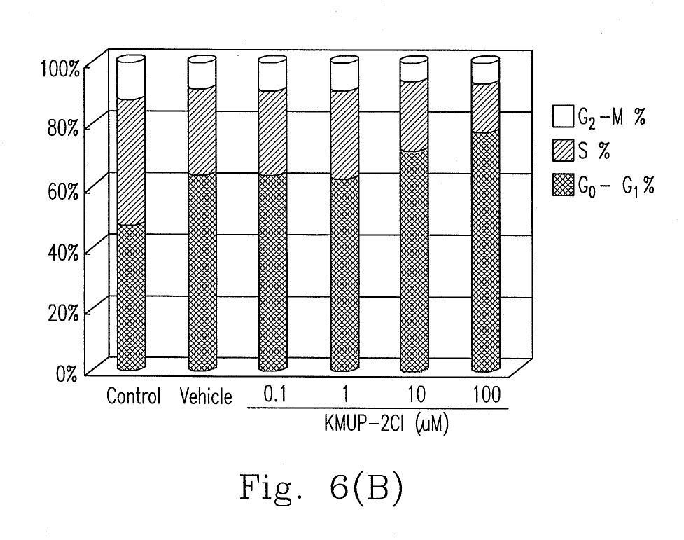

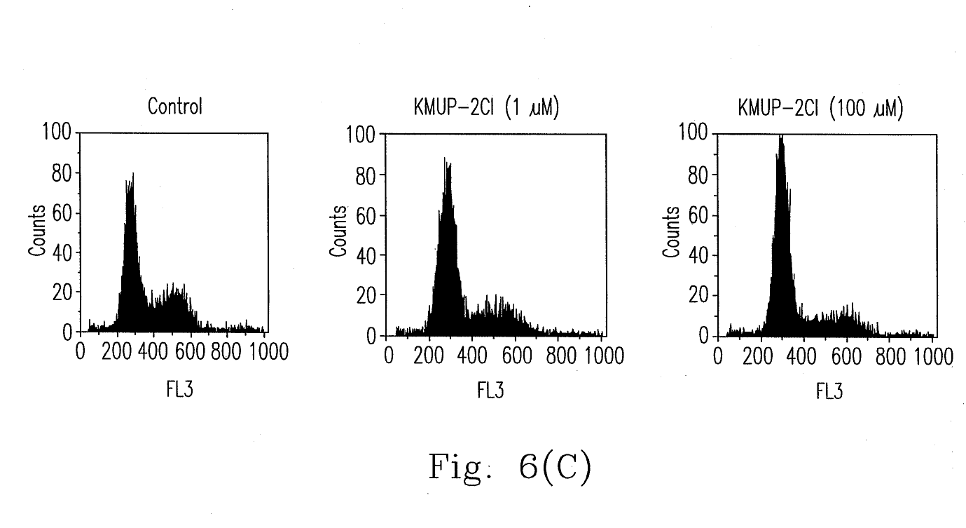

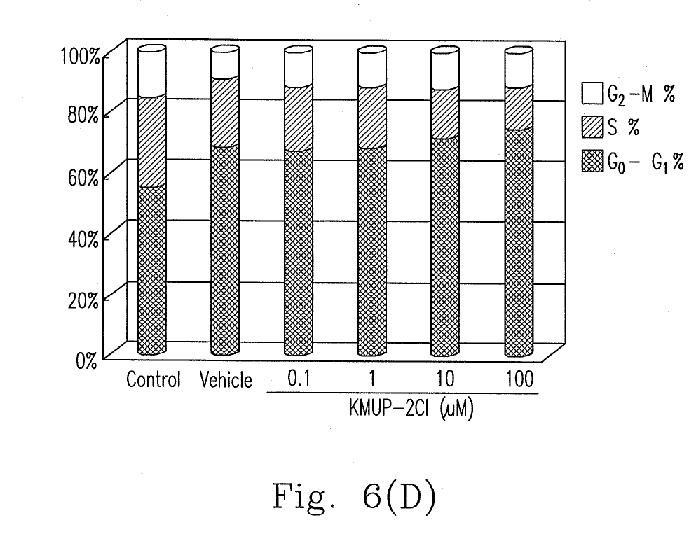

Please refer to FIGS. 6(A) and 6(D), which illustrate the distributions of different phases of SCC-25 when the human tongue squamous carcinoma SCC-25 is treated with various concentrations of KMUP-2Cl for (A, B) 24 hours and (C, D) 48 hours respectively. In FIGS. 6(A) and 6(B), the ratio in G₀/G₁ phase of SCC-25 increases from 62.85% to 77.95%, and the ratio in S phase decreases relatively (from 28.25% to 15.45%) when SCC-25 cells are treated with 1 μM and 100 μM KMUP-2Cl respectively for 24 hours. From the bar chart of FIG. 6(B), it is found that the ratio in G₀/G₁ phase increases with the increased concentrations of KMUP-2Cl, and the ratio in S phase decreases therewith. Furthermore, in FIGS. 6(C) and 6(D), the ratio in G₀/G₁ phase of SCC-25 increases from 68% to 74.95%, and the ratio in S phase thereof decreases relatively (from 20.15% to 13.45%) when SCC-25 cells are treated with 1 μM and 100 μM KMUP-2Cl respectively for 48 hours. From the bar chart of FIG. 6(D), it is found that the ratio in G₀/G₁ phase increases with the increased concentrations of KMUP-2Cl, and the ratio in S phase decrease therewith. From FIGS. 6(A) to 6(D), it can be confirmed that KMUP-2Cl ceases the cell cycle of SCC-25 in G₀/G₁ phase.

From FIGS. 1 to FIGS. 6(A)˜6(D), it is known that the prepared halogenated xanthine derivative, KMUP-2Cl, of the present invention can inhibit the growth of the human normal prostate cell line, the human prostate carcinoma, the human tongue squamous carcinoma, the human colon adenocarcinoma and the human lung carcinoma. The higher concentration of KMUP-2Cl makes the better growth inhibition. Further, KMUP-2Cl can cease the growth of the human tongue squamous carcinoma SCC-25 in G₀/G₁ phase.

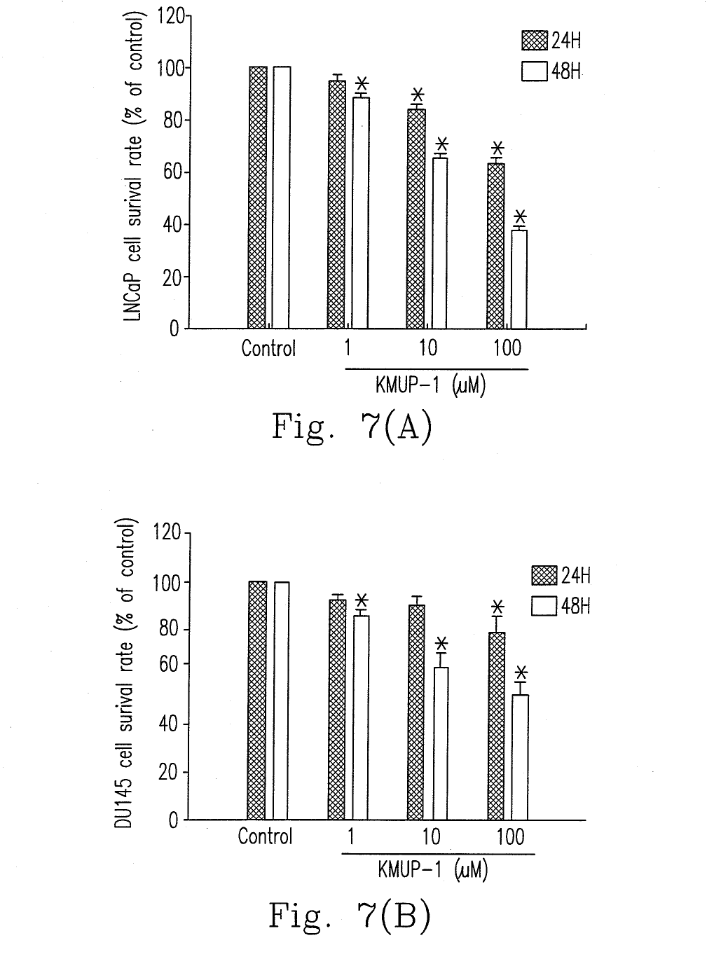

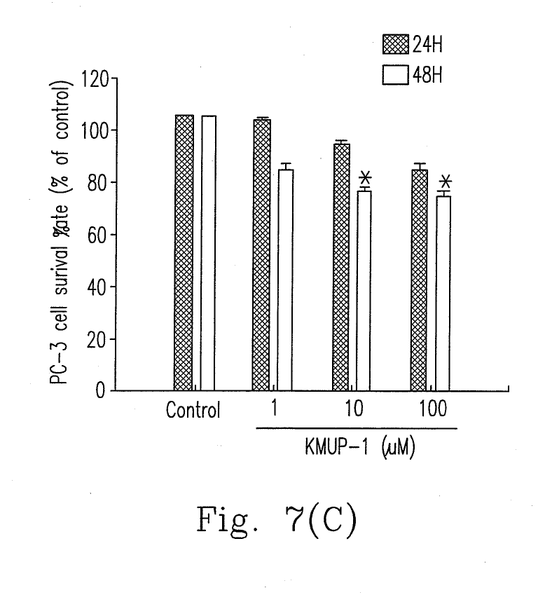

Please refer FIGS. 7(A) to 7(C), which illustrate the relationships between the survival rate and various concentrations of KMUP-1 while the human prostate carcinomas (A) LNCaP, (B) DU145 and (C) PC-3 respectively treated with various concentrations of KMUP-1 for 24 and 48 hours. In FIGS. 7(A) to 7(C), the survival rates (relative to the control) of the three carcinoma cell lines (i.e. LNCaP, DU145 and PC-3) decrease with the increased concentrations of KMUP-1, and the survival rate in 48-hour KMUP-1-treatment is significantly lower than that in 24-hour KMUP-1-treatment. It means that KMUP-1 has significant growth inhibition on these three human prostate carcinoma cell lines. The higher concentration of KMUP-1 makes more significant growth inhibition.

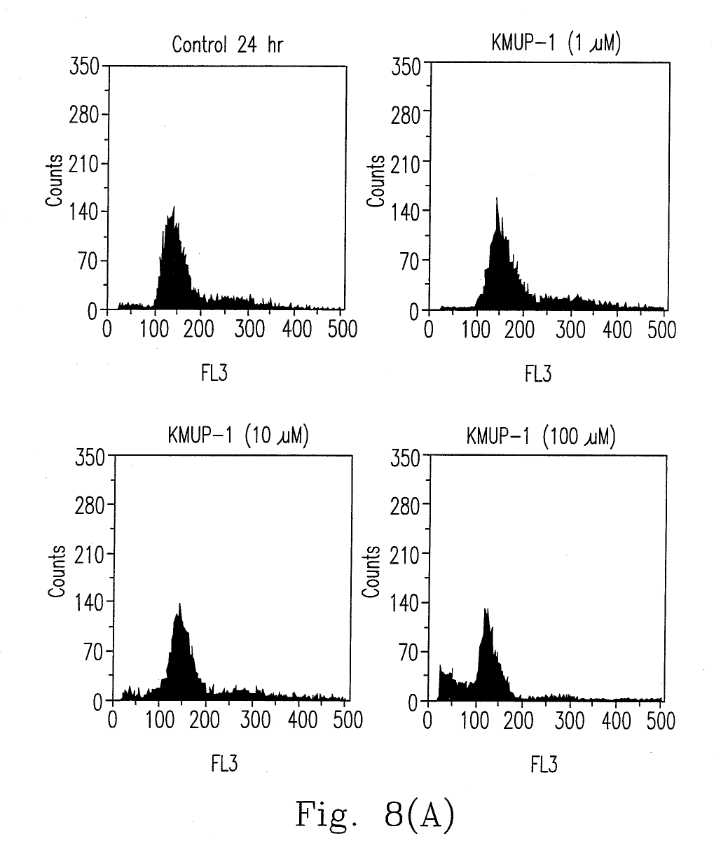

Please refer to FIGS. 8(A) and 8(B), which illustrate the distributions of the cell cycle in LNCaP while the human prostate carcinoma LNCaP is treated with various concentrations of KMUP-1 for (A) 24 hours and (B) 48 hours respectively. In FIG. 8(A), the increased concentrations of KMUP-1 react for 24 hours to inhibit the growth of LNCaP, and make the cell cycle of LNCaP cease in G₀/G₁ phase and tend to sub-G₁ phase. In FIG. 8(B), the increased concentrations of KMUP-1 react for 48 hours to inhibit the growth of LNCaP, and make the cell cycle of LNCaP cease in G₀/G₁ phase and tend to sub-G₁ phase. Therefore, KMUP-1 can inhibit the growth activity of LNCaP cells.

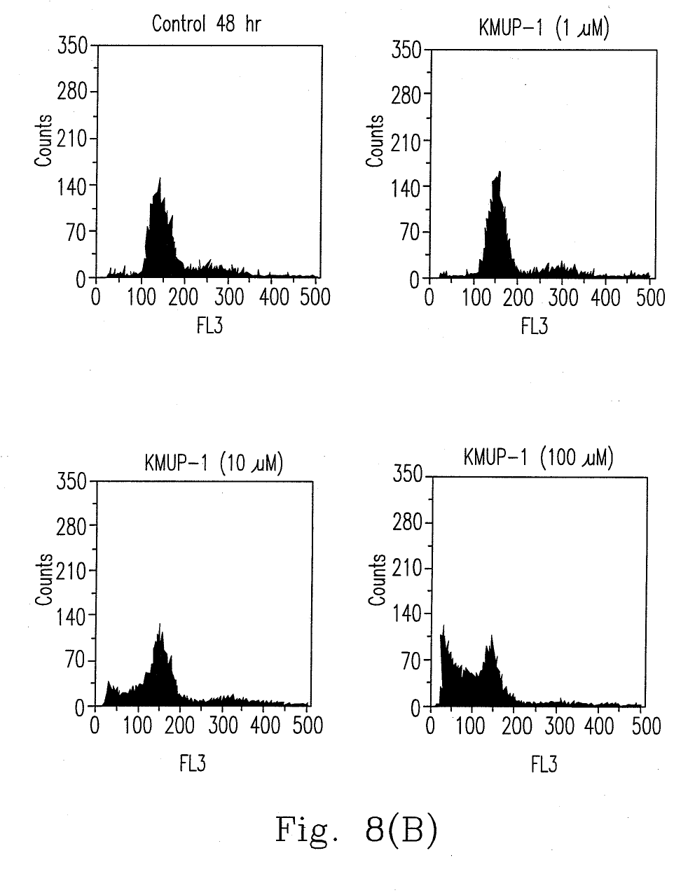

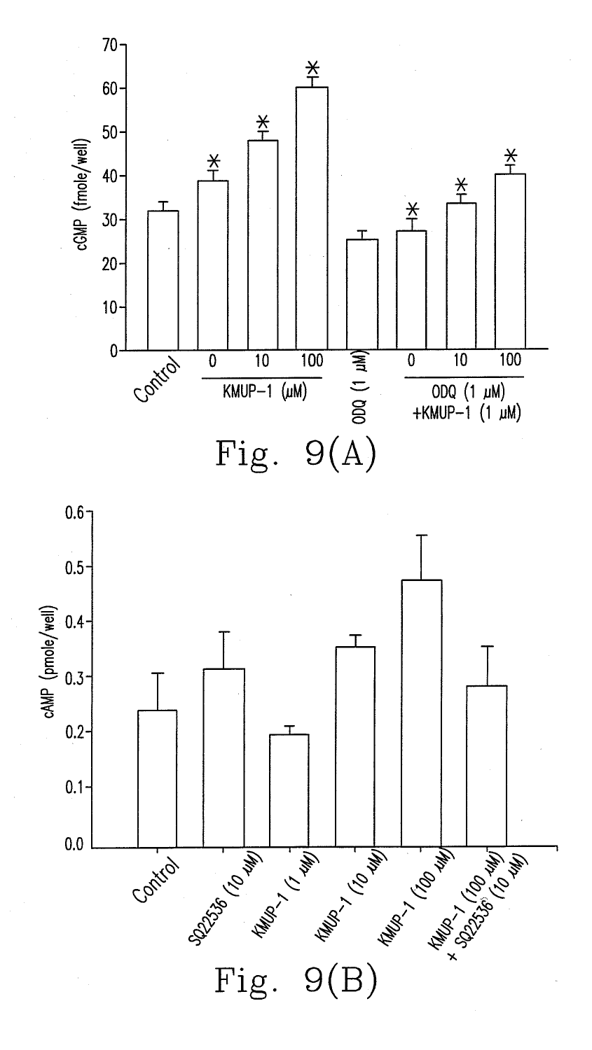

Please refer to FIG. 9(A), which illustrates the relationships between drug concentrations and the cGMP (3′,5′-cyclic guanosine monophosphate) concentration, in which the human prostate epithelial carcinoma LNCaP is treated with various concentrations of KMUP-1 individually for 48 hours, and treated with 1 μM solubale guanylyl cyclase (sGC) inhibitor ODQ and various concentrations of KMUP-1 together for 48 hours. In FIG. 9(A), as illustrated in the “Background of the invention” section of the present invention, KMUP-1 increases the content of cGMP, and higher concentration of KMUP-1 makes the higher amount of cGMP in LNCaP cells. Further, ODQ (1H-[1,2,4]oxadiazolo[4,3-a]quinoxalin-1-one) is a sGC inhibitor, and ODQ and KMUP-1 are antagonists to each other. Although ODQ and KMUP-1 can cooperate to increase the amount of cGMP on LNCaP cells, the increased effect is not significant as KMUP-1 individually functioned on LNCaP cells.

Please refer to FIG. 9(B), which illustrates the relationships between the drug concentrations and the cAMP (cyclic adenosine monophophate) concentration, in which the human prostate epithelial carcinoma LNCaP is treated with 10 μM sGC inhibitor SQ22536, various concentrations of KMUP-1, and 100 μM KMUP-1 plus 10 μM sGC inhibitor SQ22536 respectively for 48 hours. SQ22536 (9-(tetrahydro-2-(uranyl)-9H-purin-6-amine) is an adenylyl cyclase inhibitor. In FIG. 9(B), KMUP-1 increases the amount of cAMP in LNCaP cells, and the higher concentration of KMUP-1 makes the higher amount of cAMP. The amount of cAMP that SQ22536 and KMUP-1 co-function on LNCaP cells is almost equal to that of cAMP that SQ22536 co-functions on LNCaP cells. Further, while comparing the co-functions of SQ22536 and 100 μM KMUP-1 with the individual function of 100 μM KMUP-1, it is found that the co-functions of SQ22536 and 100 μM KMUP-1 can decrease the amount of cAMP in LNCaP cells. It means that SQ22536 also can antagonize the improved effect of KMUP-1.

Therefore, from the FIGS. 9(A) and 9(B), it is known that KMUP-1 can increase the amount of cAMP/cGMP in the cells, and KMUP-1 also can be inhibited by the sGC inhibitor ODQ and adenylyl cyclase inhibitor SQ22536.

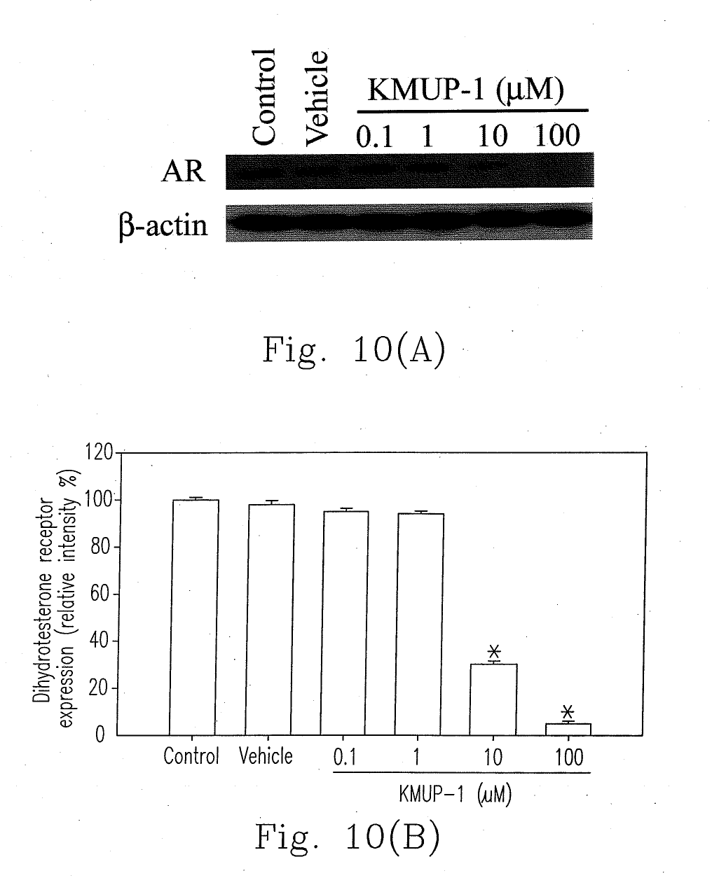

Please refer to FIGS. 10(A) and 10(B), which illustrate (A) the immunoblotting analysis of AR (androgen receptor) and (B) the relationship between the drug concentrations and dihydrotestosterone receptor, in which the human prostate epithelial carcinoma LNCaP is treated with various concentrations of KMUP-1 and KMUP-1 vehicle for 48 hours. From FIGS. 10(A) and 10(B), KMUP-1 can inhibit the expressions of AR and dihydrotestosterone receptor. The AR expression is completely inhibited, and the dihydrotestosterone receptor expression is also relatively low when KMUP-1 concentration is 100 μM.

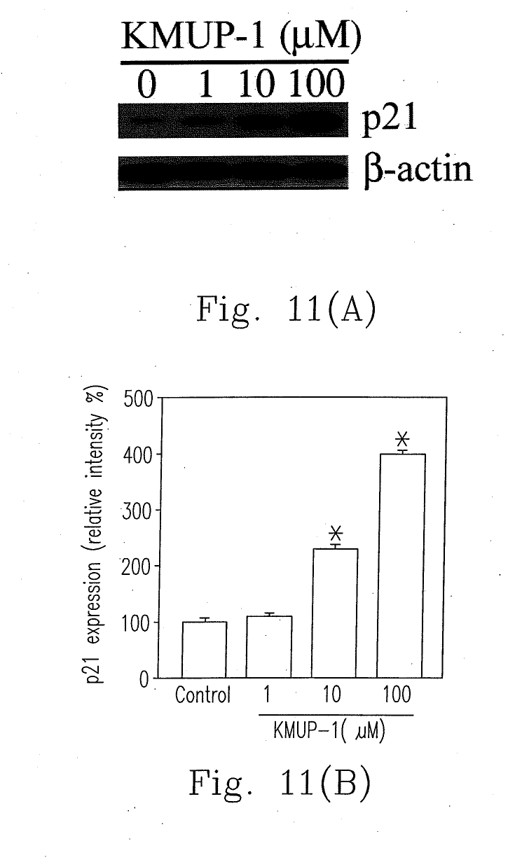

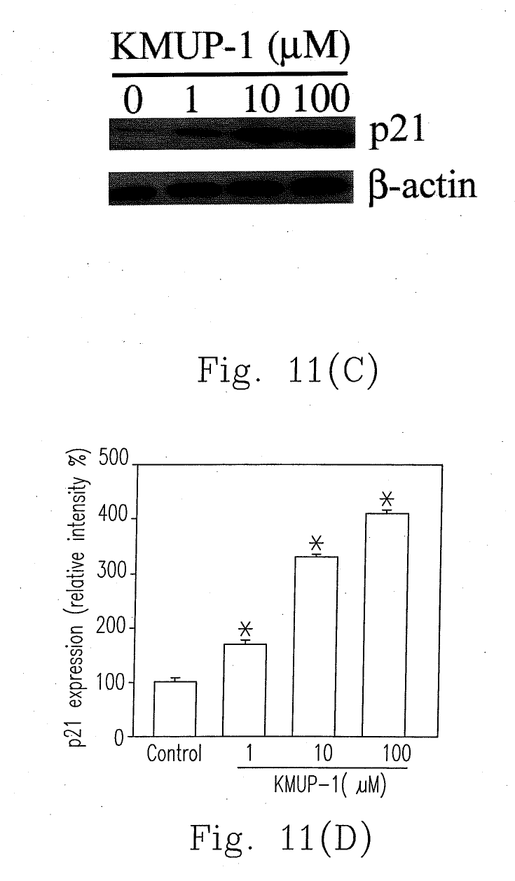

Please refer to FIGS. 11(A) to 11(D), which illustrate (A, C) the immunoblotting analysis of p21 protein (A: 24 hours; C: 48 hours) and (B, D) the relationship between the relative intensity of p21 expression and various concentrations of KMUP-1 (B: 24 hours; D: 48 hours), in which the human prostate epithelial carcinoma LNCaP is treated with various concentrations of KMUP-1 for 24 and 48 hours respectively. Cyclin-dependent kinase inhibitor 1A (CDKN1A, also nominated as p21) combines with the cyclin-CDK2 and cyclin-CDK4 complex, inhibits the activity of cyclin-CDK2 and cyclin-CDK4 complex, and p21 also regulates G₁ phase. From FIGS. 11(A) and 11(C), p21 expression would be increased by reacting KMUP-1 on LNCaP cells for 24 and 48 hours. The higher concentration of KMUP-1 makes the higher p21 expression. The p21 expression at 100 μM of KMUP-1 is four times to that at 1 μM of KMUP-1.

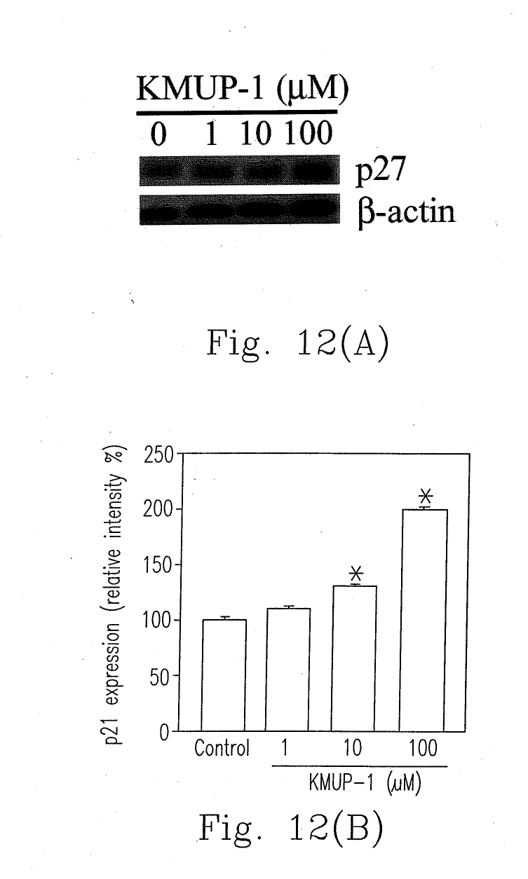

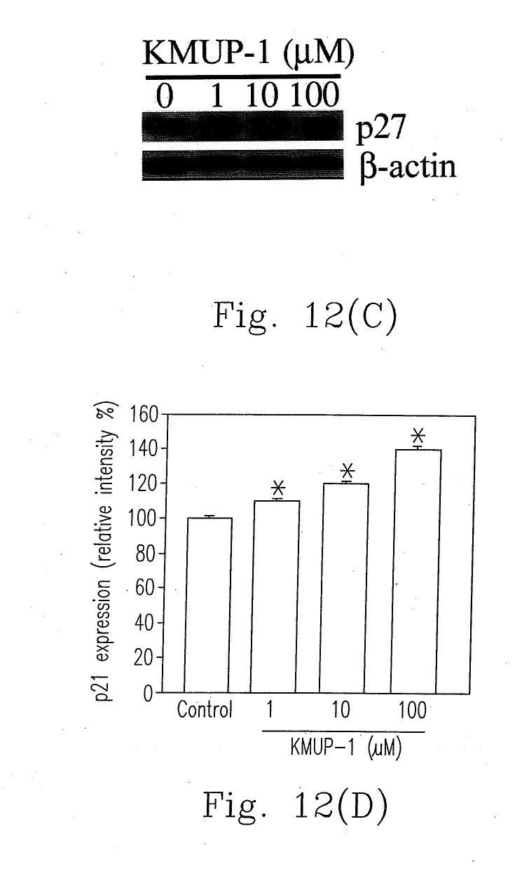

Please refer to FIG. 12(A) to 12(D), which illustrate (A, C) the immunoblotting analysis of p27 protein (A: 24 hours; C: 48 hours) and (B, D) the relationship between the relative intensity of p27 expression and various concentrations of KMUP-1 (B: 24 hours; D: 48 hours), in which the human prostate epithelial carcinoma LNCaP is treated with various concentrations of KMUP-1 for 24 and 48 hours respectively. p27 belongs to the Cip/Kip family of Cyclin-dependent kinase inhibitor 1B (CDKN1B, also nominated as p27)-inhibited proteins. p27 usually is a cell cycle inhibition protein since its function is to cease or slow down the mitosis. The trend of p27 expression in FIGS. 12(A) to 12(D) is similar with that of p21 expression, and it means that p27 expression would be increased by reacting KMUP-1 on LNCaP cells for 24 and 48 hours. In particular, the p27 expression at 100 μM of KMUP-1 is twice to that at 1 μM of KMUP-1 while LNCaP cells are treated with KMUP-1 for 24 hours.

p27 can combine with Cyclin D, or combine with the catalytic subunit CDK4 of Cyclin D to form a complex. p27 can inhibit the catalytic activity of CDK4. The increased amount of p27 would induce the cell cycle to stop in G₁ phase. Identically, p27 also can combine with other CDK proteins while p27 and other Cyclin subunit (such as Cyclin E/CDK2 and Cyclin A/CDK1) forms the complex.

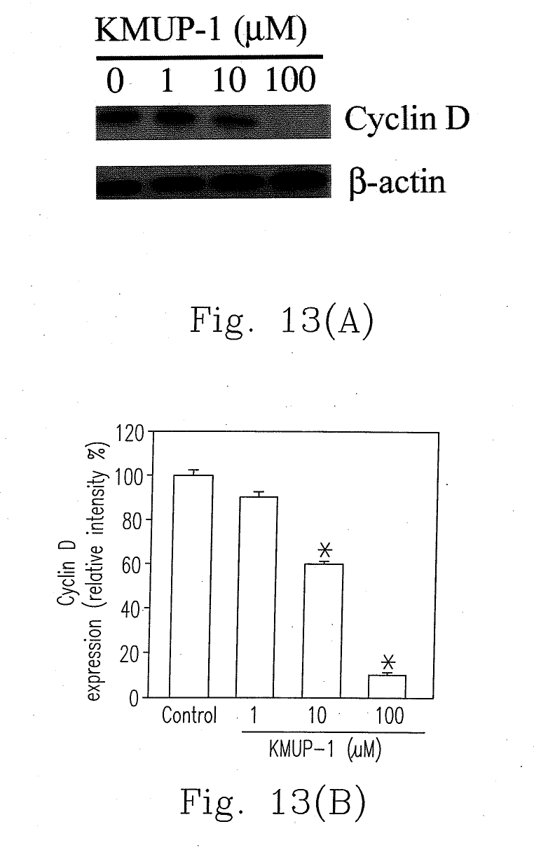

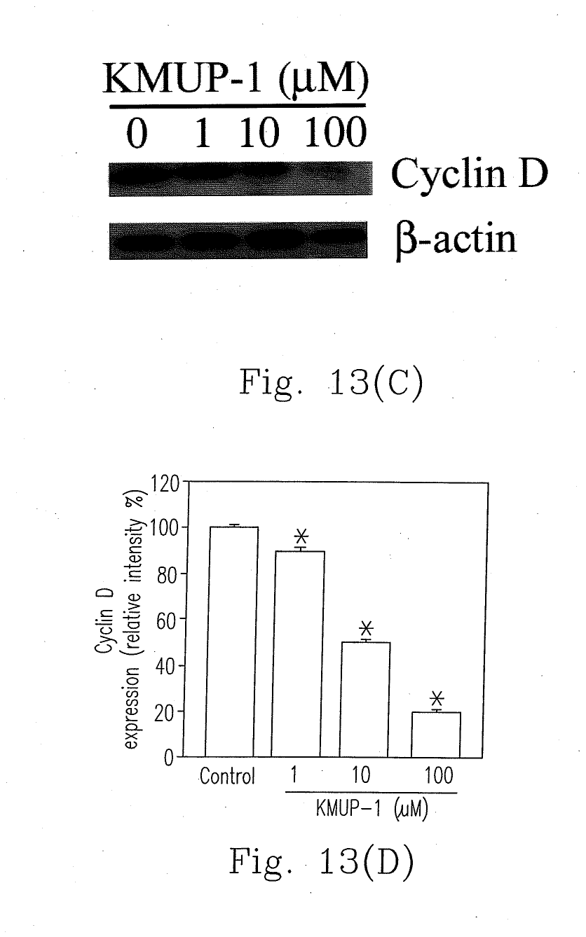

Please refer to FIGS. 13(A) to 11(D), which illustrate (A, C) the immunoblotting analysis of Cyclin D protein (A: 24 hours; C: 48 hours) and (B, D) the relationship between the relative intensity of Cyclin D expression and various concentrations of KMUP-1 (B: 24 hours; D: 48 hours), in which the human prostate epithelial carcinoma LNCaP is treated with various concentrations of KMUP-1 for 24 and 48 hours respectively. From FIGS. 13(A) to 13(D), it is known that Cyclin D expression would be decreased by reacting KMUP-1 on LNCaP cells for 24 and 48 hours. The higher concentration of KMUP-1 makes the lower Cyclin D expression. Cyclin D expression at 100 μM of KMUP-1 is one ninth to that at 1 μM of KMUP-1 while LNCaP cells are treated with KMUP-1 for 24 hours. Similarly, Cyclin D expression at 100 μM of KMUP-1 is 20% to that at 1 μM of KMUP-1 while LNCaP cells are treated with KMUP-1 for 48 hours.

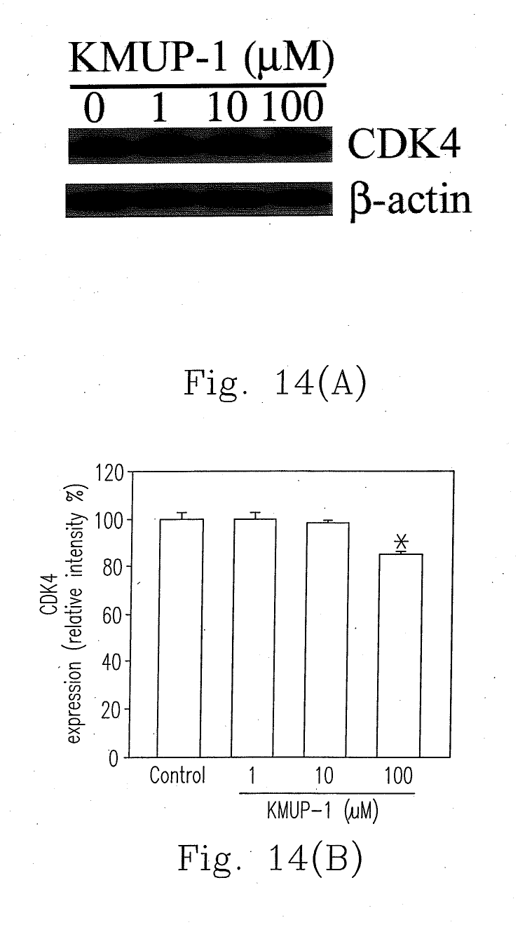

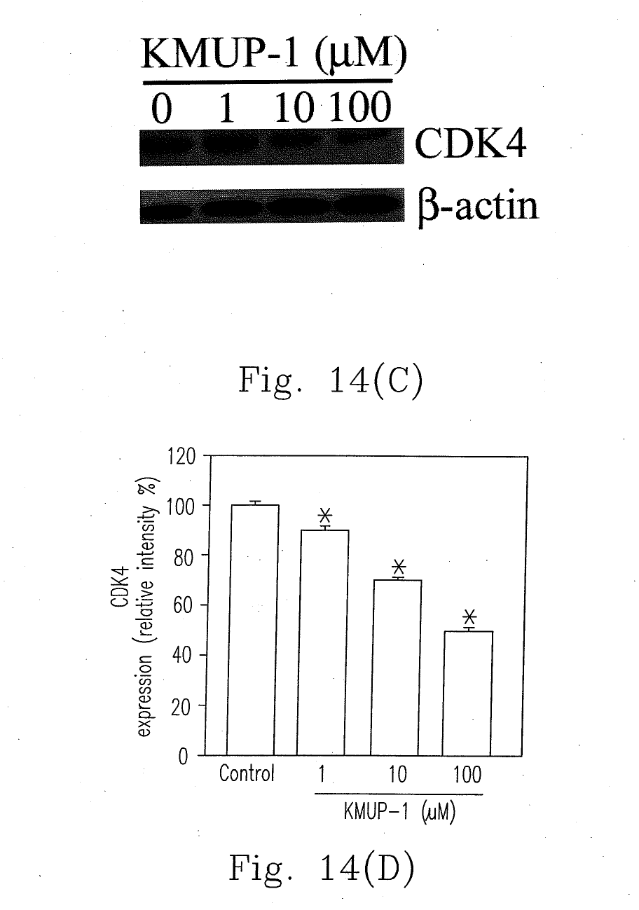

Please refer to FIG. 14(A) to 14(D), which illustrate (A, C) the immunoblotting analysis of CDK4 protein (A: 24 hours; C: 48 hours) and (B, D) the relationship between the relative intensity of CDK4 expression and various concentrations of KMUP-1 (B: 24 hours; D: 48 hours), in which the human prostate epithelial carcinoma LNCaP is treated with various concentrations of KMUP-1 for 24 and 48 hours respectively. From FIGS. 14(A) to 14(D), it is known that CDK4 expression would be decreased by reacting KMUP-1 on LNCaP cells for 24 and 48 hours. The higher concentration of KMUP-1 makes the lower CDK4 expression. CDK4 expression at 100 μM of KMUP-1 is 50% to that at 1 μM of KMUP-1 while LNCaP cells are treated with KMUP-1 for 48 hours.

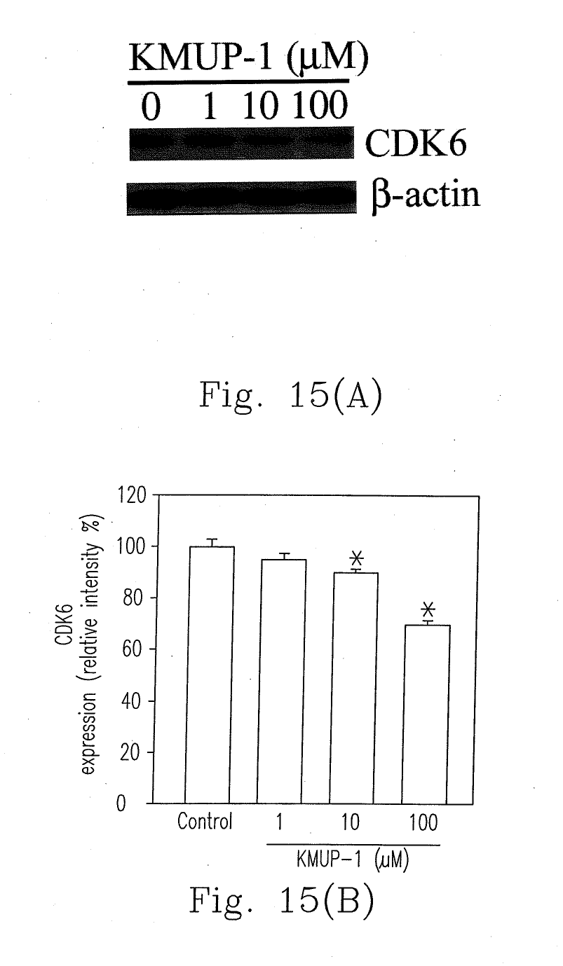

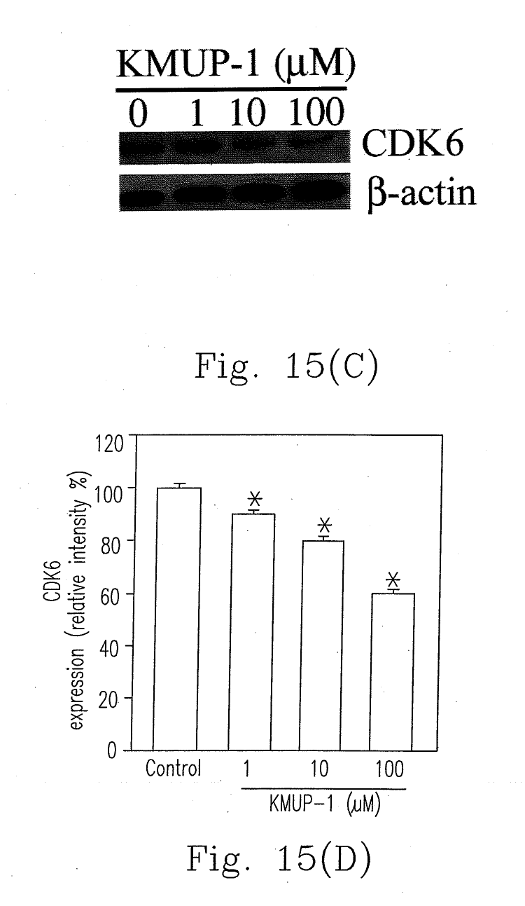

Please refer to FIG. 15(A) to 15(D), which illustrate (A, C) the immunoblotting analysis of CDK6 protein (A: 24 hours; C: 48 hours) and (B, D) the relationship between the relative intensity of CDK6 expression and various concentrations of KMUP-1 (B: 24 hours; D: 48 hours), in which the human prostate epithelial carcinoma LNCaP is treated with various concentrations of KMUP-1 for 24 and 48 hours respectively. From FIGS. 15(A) to 15(D), it is known that CDK6 expression would be decreased by reacting KMUP-1 on LNCaP cells for 24 and 48 hours. The higher concentration of KMUP-1 makes the lower CDK6 expression. CDK6 expression of 48-hour reaction is lower than that of 24-hour reaction without regarding to the concentration of KMUP-1.

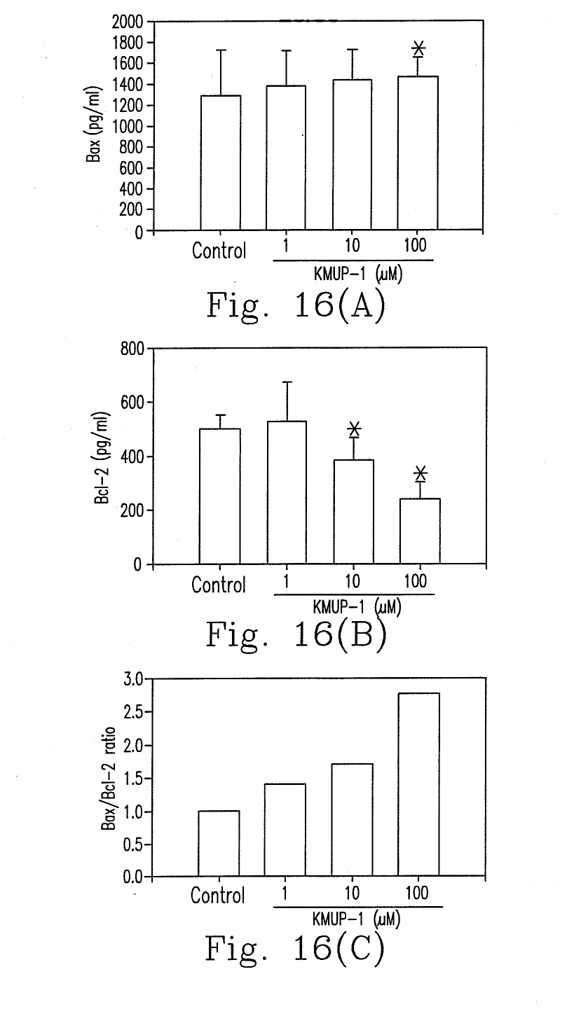

Please refer to FIGS. 16(A) to 16(C), which illustrate the relationships between the various concentrations of KMUP-1 and (A) Bax expression, (B) Bcl-2 expression, and (C) the Bax/Bcl-2 ratio, in which the human prostate epithelial carcinoma LNCaP is treated with various concentrations of KMUP-1 for 48 hours. In FIG. 16(A), Bax expression in LNCaP cells has no difference with the increased concentration of KMUP-1. In FIG. 16(B), Bcl-2 expression in LNCaP cells decreases with the increased concentration of KMUP-1. Bax expression and Bcl-2 expression is represented by the Bax/Bcl-2 ratio as shown in FIG. 16(C), and it is found that the Bax/Bcl-2 ratio in LNCaP cells increases with the increased concentration of KMUP-1.

From FIGS. 7(A) and 7(B) to FIGS. 16(A)˜16(C), it is known that KMUP-1 can inhibit the growth of human prostate epithelial carcinomas LNCaP, DU145 and PC-3. Furthermore, KMUP-1 also can cease the growth of human prostate epithelial carcinoma LNCaP in G₀/G₁ phase. KMUP-1 can improve the amounts of cGMP and cAMP in LNCaP cells, inhibit androgen receptor expression, and induce p21 and p27 expressions in the cell signaling transduction. Since p21 is a cyclin-CDK4 inhibitor, p27 also can be the CDK inhibitor. Therefore, p21 and p27 are activated while LNCaP cells are treated with KMUP-1, p21 and p27 further inhibit CDK4, CDk6 and Cyclin D expressions, and mitosis of LNCaP cells are ceased or slowed down.

In addition to observe the inhibition effect of the halogenated xanthine derivatives on the cancer cell lines in the cellular level, the animal experiments are further performed. LNCaP-xenografted rude mice are injected intraperitoneally with KMUP-1 (150 and 150 mg/kg) and dosed orally with KMUP-1 (200 and 400 mg/kg) respectively once per day, and a continuity for two months. Comparing with the control, the intraperitoneal injection of KMUP-1 can significantly eliminate the LNCaP prostate tumor in the xenografted rude mice. The significant effect can be obtained (data not shown) when KMUP-1 is 100 mg/kg. The oral dosage of KMUP-1 also can significantly eliminate the LNCaP prostate tumor in the xenografted rude mice. The significant effect can be obtained (data not shown) when KMUP-1 is 200 mg/kg.

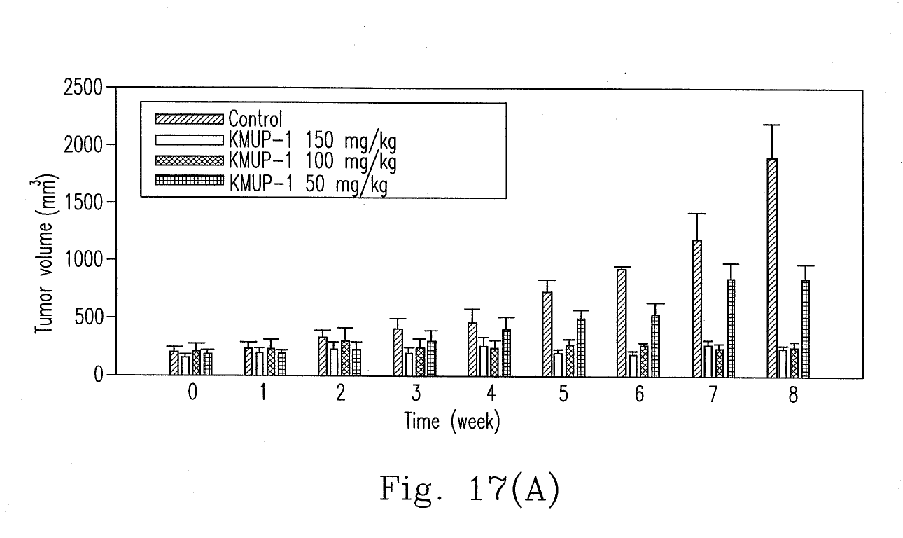

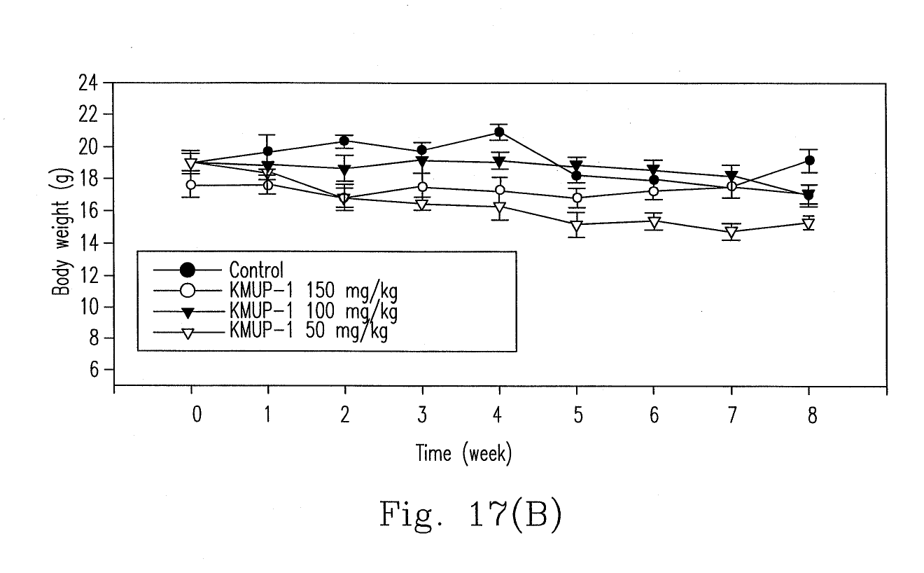

Please refer to FIG. 17(A), which illustrates the relationship between the tumor size and the experimental time (week), in which LNCaP-xenografted rude mice are injected subcutaneously with KMUP-1. In FIG. 17(A), the higher concentration of KMUP-1 (100 and 150 mg/kg) has stronger ability to inhibit LNCaP prostate tumor size which does not enlarge anymore. However, placebo (control) cannot inhibit LNCaP prostate tumor size if the placebo is intraperitoneally injected therein. Even, the tumor size after subcutaneously injecting placebo enlarges for four times to that prior to the experiments.

Please refer to FIG. 17(B), which illustrate the relationship between the body weight and the experimental time (week), in which LNCaP-xenografted rude mice are injected subcutaneously with KMUP-1. In FIG. 17(B), subcutaneous injection with KMUP-1 can decrease the body weight of LNCaP-xenografted rude mice. However, the body weight of LNCaP-xenografted rude mice which are subcutaneously injected with the control (placebo) will be slightly increased.

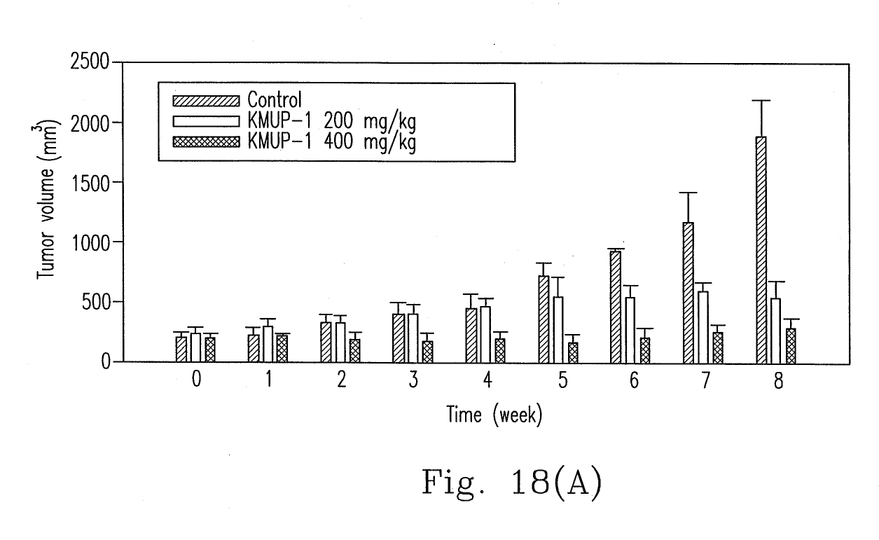

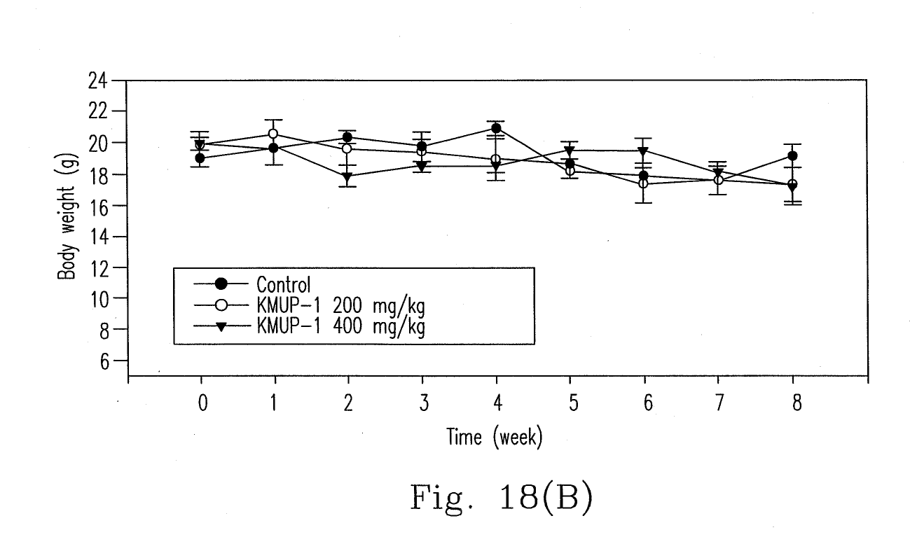

Please refer to FIG. 18(A), which illustrates the relationship between the tumor size and the experimental time (week), in which LNCaP-xenografted rude mice are orally dosed with KMUP-1. In FIG. 18(A), the higher concentration of KMUP-1 (400 mg/kg) has stronger ability to inhibit LNCaP prostate tumor size which does not enlarge anymore. However, the placebo (control) cannot inhibit LNCaP prostate tumor size if the placebo is orally dosed therein. Even, the tumor size after orally dosing the placebo enlarges for four times to that prior to the experiments.

Please refer to FIG. 18(B), which illustrate the relationship between the body weight and the experimental time (week), in which LNCaP-xenografted rude mice are dosed orally with KMUP-1. In FIG. 18(B), the body weights of the LNCaP-xenografted rude mice in the experimental group and the control group do not have significant difference after dosing orally KMUP-1 or the control (placebo) for eight weeks.

From FIGS. 17(A) and 17(B) to FIGS. 18(A) and 18(B), it is known that oral form or subcutaneously injection of KMUP-1 can all effectively inhibit the tumor size of LNCaP-xenografted rude mice, and the body weights of rude mice do not obviously increase or decrease to prevent other sequelae.

Accordingly, the xanthine derivatives and the halogenated xanthine derivatives prepared in the present invention can effectively inhibit the growth and the metastasis of carcinoma in vitro, and can effectively inhibit the growth of prostate tumor and even decrease the tumor size in vivo. In particular, the xanthine derivatives and the halogenated xanthine derivatives of the present invention have significant inhibition effect on cell lines from different human tissues.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A halogenated xanthine derivative having a structure as shown in the following formula I: 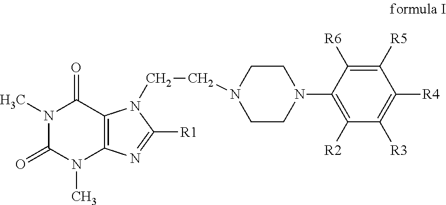
wherein R1 represents a first substituted group selected from a group consisting of a first hydrogen group, a first halide group and a C₁-C₁₀ alkyl group, and each of R2, R3, R4, R5 and R6 represents a second substituted group being one of a second hydrogen group and a second halide group, wherein each of the first halide group and the second halide group has one halide atom selected from a group consisting of a chloride (Cl), a bromide (Br) and an iodine (I).

2. The derivative according to claim 1, wherein R1, R2, R3, R4, R5 and R6 have an identical radioactive halide group.

3. The derivative according to claim 2, wherein the identical radioactive halide group is one radioactive halide atom selected from a group consisting of a radioactive chloride, a radioactive bromide and a radioactive iodine.

4. The derivative according to claim 1, wherein R1 R2, R3, R4, R5 and R6 are different radioactive halide groups.

5. The derivative according to claim 4, wherein each of the different radioactive halide groups is one radioactive halide atom selected from a group consisting of a radioactive chloride, a radioactive bromide and a radioactive iodine.

6. The derivative according to claim 1, wherein each of R1, R2, R3, R4, R5 and R6 is a radioactive halide group selected from a group consisting of a ³⁸Cl, a ³⁷Cl, a ⁷⁵Br, a ⁷⁶Br, a ⁷⁷Br, a ⁸²Br, a ¹²²I, ¹²³I, an ¹²⁴I, an ¹²⁵I and ¹³¹I.

7. The derivative according to claim 1 having a function selected from a group of inhibiting a cancer, inhibiting a cancer metastasis and a combination thereof.

8. A method for preparing a halogenated xanthine derivative having a structure as shown in the following formula II: 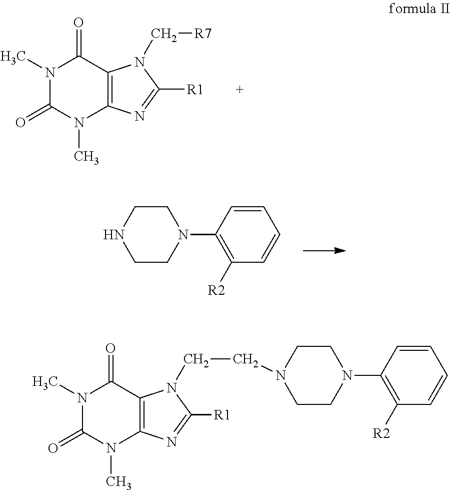
comprising a step of:
(a) reacting a theophylline with a piperazine in a first solution to obtain the halogenated xanthine derivative,
wherein R1 represents a first substituted group being one of a first hydrogen group and a first halide group, R7 represents a second substituted group being one of a second hydrogen group and a second halide group, and R2 represents a third substituted group being one of a third hydrogen group and a third halide group.

9. The method according to claim 8, further comprising a step of
(b) radio-labeling a radioactive halogen group from a radioactive halide compound on at least one of R1, R2 and R7 of the halogenated xanthine derivative to obtain a radioactive halogenated xanthine derivative.

10. The method according to claim 9, wherein the radioactive halide compound is one of a radioactive halogen, a radioactive sodium halide and a combination thereof which are dissolved in a second solution.

11. The method according to claim 10, wherein the second solution is an organic solvent selected from a group consisting of a tetrahydrofuran, a methanol and an ethanol.

12. The method according to claim 8 wherein the first solution is an organic solvent selected from a group consisting of a tetrahydrofuran, a methanol and an ethanol.

13. A precursor of a halogenated xanthine derivative having a structure as shown in the following formula III: 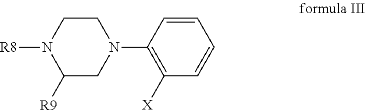
wherein R8 represents a first substituted group selected from a group consisting of a first hydrogen group, a first C₁-C₁₂ alkyl group, a first C₁-C₁₂ alkenyl group and a first C₁-C₁₂ dihaloalkyl group, R9 represents a second substituted group selected from a group consisting of a second hydrogen group, a second C₁-C₁₂ alkyl group, a second C₁-C₁₂ alkenyl group, a monohaloalkyl group and a second C₁-C₁₂ dihaloalkyl group, and X represents one of a halide group and a radioactive halide group.

14. The precursor according to claim 13, wherein the halide group is a halide atom selected from a group consisting of a chloride (Cl), a bromide (Br) and an iodine (I), and the radioactive halide group is a radioactive halide atom selected from a group consisting of a radioactive chloride, a radioactive bromide and a radioactive iodine.

15. A precursor of a halogenated xanthine derivative having a structure as shown in the following formula IV: 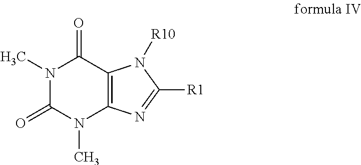
wherein R1 represents a first substituted group being one of a hydrogen group and a halide group, and R10 represents a C₁-C₁₂ iodoalkyl group.

16. The precursor according to claim 15, wherein the halide group is one halide atom selected from a group consisting of a chloride (Cl), a bromide (Br) and an iodine (I).

17. The precursor according to claim 15 being obtained by reacting a dihaloalkane with one of a theophylline and a chlorotheophylline.

18. The precursor according to claim 15, wherein an iodide group of the C₁-C₁₂ iodoalkylgroup of the precursor is further radio-labeled a first radioactive halide group.

19. The precursor according to claim 18, wherein the halide group is further radio-labeled a second radioactive halide group when R1 of the precursor is the halide group.

20. The precursor according to claim 15, wherein the precursor is an iodomethyl chlorotheophylline when R1 is the chloride group and R10 is an iodomethyl group.
